(19) United States
(12) Patent Application Publication
(10) Pub. No.: US 2026/0283677 A1
(43) Pub. Date: Sep. 24, 2026

(54) INTRAOSSEOUS ACCESS DEVICE WITH RELIEVED DISTAL CURVE FOR IMPROVED SUPPORT AND FLEXIBILITY

(71) Applicant: Boston Scientific Scimed, Inc., Maple Grove, MN (US)

(72) Inventors: Neil Shigeo Sasaki, San Jose, CA (US); Benjamin David Parker, Sunnyvale, CA (US); Brian W. Donovan, San Jose, CA (US); Michael Willink, Sunnyvale, CA (US)

(21) Appl. No.: 19/572,465

(22) Filed: Mar. 19, 2026

Related U.S. Application Data
(60) Provisional application No. 63/775,138, filed on Mar. 20, 2025.

(51) Int. Cl. A61B 18/14; A61B 17/34; A61B 18/12; A61B 18/00

(52) U.S. Cl. CPC A61B 18/14; A61B 17/3423; A61B 17/3472; A61B 18/1206; A61B 2018/00184; A61B 2018/00339; A61B 2018/00565; A61B 2018/126

(57) ABSTRACT

A medical device for forming a channel into bone includes an introducer cannula having an introducer lumen disposed therethrough and a curved cannula having an elongated tube with a curved cannula lumen. The elongated tube includes a proximal straight portion and a distal curved portion configured to be received into the introducer lumen. A stylet having an elongated shaft with a proximal straight portion and a distal curved portion is configured to be received into the curved cannula lumen. The distal curved portion of the stylet comprises relief structures along an outer curvature configured to maintain a consistent cross-sectional profile during deflection. The relief structures comprise uniformly spaced projections and recesses with radially-oriented ridges configured to support a curved cannula wall during deflection. The ridges have filleted edges to facilitate smooth insertion and removal through the curved cannula while maintaining structural integrity during bone penetration.

### CROSS REFERENCE TO RELATED APPLICATIONS

This application claims priority to U.S. Provisional Application No. 63/775,138 entitled “INTRAOSSEOUS ACCESS DEVICE WITH RELIEVED DISTAL CURVE FOR IMPROVED SUPPORT AND FLEXIBILITY,” filed Mar. 20, 2025, which is hereby incorporated by reference in its entirety.

### TECHNICAL FIELD

Described herein are various implementations of systems and devices for accessing and/or modulating tissue. Systems or kits of access tools for accessing target treatment locations within vertebral bodies are also provided. The access tools may include, for example, a curved J-stylet configured for easy removal from a curved cannula while still providing sufficient curved trajectory to reach a target treatment location within vertebral bodies at varying vertebral levels of the spine and having varying bone density.

### BACKGROUND

Back pain is a very common health problem worldwide and is a major cause for work-related disability benefits and compensation. At any given time, low back pain impacts nearly 30% of the US population, leading to 62 million annual visits to hospitals, emergency departments, outpatient clinics, and physician offices. Back pain may arise from strained muscles, ligaments, or tendons in the back and/or structural problems with bones or spinal discs. The back pain may be acute or chronic. Existing treatments for chronic back pain vary widely and include physical therapy and exercise, chiropractic treatments, injections, rest, pharmacological therapy such as opioids, pain relievers or anti-inflammatory medications, and surgical intervention such as vertebral fusion, discectomy (e.g., total disc replacement), or disc repair. Existing treatments can be costly, addictive, temporary, ineffective, and/or can increase the pain or require long recovery times. In addition, existing treatments do not provide adequate relief for the majority of patients and only a small percentage are surgically eligible.

Access to vertebral bodies for treatment presents challenges, particularly when navigating through varying bone densities and anatomical structures. Current access tools may face difficulties maintaining consistent performance across different bone densities, can generate unwanted particulate during insertion and removal, and may not provide optimal control during curved trajectories. Additionally, existing curved access instruments may experience deformation or profile changes during use, potentially affecting their reliability and performance. These challenges may be particularly evident when accessing regions of vertebral bodies where precise navigation and maintained geometry are needed for successful treatment.

### SUMMARY

Applicant’s existing technology (the Intracept^{®} procedure by Relievant^{®}) offers a safe and effective minimally invasive procedure that targets the basivertebral nerve for the relief of chronic vertebrogenic low back pain. As disclosed herein, several embodiments provide bone access tools, additional modalities of relief for patients and/or adjunct technologies.

In accordance with several embodiments, an introducer system may include an introducer assembly comprising an introducer cannula and an introducer stylet. The introducer stylet may be bevel tipped, trocar tipped, and/or diamond tipped. The introducer stylet is configured to be received in a lumen of the introducer cannula in a manner such that a distal tip of the introducer stylet protrudes from an open distal tip of the introducer cannula thereby forming the introducer assembly in combination.

In accordance with several embodiments, the introducer system may further include a curved cannula assembly. The curved cannula assembly may include a cannula comprising a proximal handle with a curved insertion slot and a distal polymeric tube. The distal polymeric tube may include a curved distal end portion having a preformed curvature but configured to transition to a generally straight configuration when placed under constraint (e.g., constraint by insertion through a straight introducer cannula). The curved cannula assembly may further include a stylet (e.g., a J-stylet) comprising a proximal handle and a distal elongate shaft. The distal elongate shaft includes a curved distal end portion having a preformed curvature but configured to transition to a generally straight configuration when placed under constraint (e.g., constraint by insertion through a cannula) and a distal channeling tip. The distal elongate shaft may also include a curved distal end portion having relief structures along an outer curvature configured to maintain a consistent cross-sectional profile during deflection. The relief structures may comprise a plurality of radially-oriented ridges uniformly spaced along the outer curvature of the distal curved portion configured to engage with and support a curved cannula wall during deflection.

In accordance with several embodiments, the curved distal end portion of the J-stylet may be constructed for easy retraction from the curved cannula when the J-stylet is fully received into the curved cannula lumen during a procedure. The curved distal end portion of the J-stylet may have a section of reduced thickness that is about 20% - 80% (e.g., 20% - 60 %, 30% - 70%, 40% - 80% or overlapping ranges thereof) of the full circumferential thickness of the distal elongate shaft of the J-stylet. The reduced thickness may be achieved by removing part of material of the curved distal end portion. In some embodiments, the curved distal end portion may have a sloped transition section between a reduced thickness section and the distal channeling tip. In some embodiments, the reduced thickness may not be uniform from a proximal end to a distal end of the curved distal end portion. The reduced thickness section may taper down from the proximal end to the distal end of the curved distal end portion. Alternatively, the reduced thickness section may taper down from the distal end to the proximal end of the curved distal end portion. In some embodiments, the curved distal end portion of the J-stylet may include a relief structure along the outer curvature of its distal curved portion. The relief structure comprises a plurality of ridges that are radially-oriented and facilitate performance across varying bone densities while enabling smooth insertion and removal. The ridges may have filleted edges to maintain smooth engagement with the curved cannula wall during advancement and retraction. The distal end portion may also comprise a sharpened distal channeling tip where the tip is a wire EDM cut (electrical discharge machining) distal channeling tip configured to enhance bone engagement. In some embodiments, the curved distal end portion may have a bend angle of from 60 degrees to 85 degrees, and the elongated shaft may have specific dimensional relationships that optimize performance, such as particular shaft thickness and ridge spacing configured to provide desired stiffness and flexibility characteristics. In some embodiments, the distal open tip of curved cannula may have a beveled or angled tip (e.g., a bevel that is substantially parallel to a bevel of a distal channeling tip of the J-stylet when the J-stylet is received into the curved cannula lumen).

In Example 1, a system for treating a vertebral body is provided. The system comprises an introducer cannula having an introducer lumen disposed therethrough, and a curved cannula having an elongated tube with a curved cannula lumen disposed therethrough. The elongated tube has a proximal straight portion and a distal curved portion, the elongated tube is configured to be received into the introducer lumen of the introducer cannula. The system further comprises a stylet having an elongated shaft with a proximal straight portion and a distal curved portion, the elongated shaft configured to be received into the curved cannula lumen. The distal curved portion of the stylet comprises relief structures uniformly spaced along an outer curvature configured to maintain a consistent cross-sectional profile during deflection and during navigation through bone. The system also comprises a treatment probe configured to be advanced through the curved cannula after removal of the stylet.

In Example 2, the system of Example 1, wherein the relief structures comprise a plurality of projections and recesses, wherein the plurality of projections and recesses are configured to create discrete flex points.

In Example 3, the system of Example 2, wherein the plurality of projections and recesses comprise a plurality of ridges radially-oriented and configured to engage with and support a curved cannula wall during deflection.

In Example 4, the system of Example 3, wherein each of the plurality of ridges has an edge profile to maintain smooth engagement with the curved cannula wall, wherein the edge profile is a filleted edge profile.

In Example 5, the system of any one of Examples 3-4, wherein each of the plurality of ridges are perpendicular to a longitudinal axis of the elongated shaft.

In Example 6, the system of any one of Examples 3-5, wherein the plurality of ridges have a depth configured to maintain structural integrity while enabling controlled bending.

In Example 7, the system of any one of Examples 1-6, wherein the distal curved portion includes a section proximal to the relief structures configured to prevent kinking.

In Example 8, the system of any one of Examples 1-7, wherein the stylet further comprises a distal channeling tip, wherein the distal channeling tip of the curved cannula has a sharper angle at the tip.

In Example 9, the system of any one of Examples 1-8, wherein the distal curved portion has a bend angle between 60 and 85 degrees.

In Example 10, the system of any one of Examples 1-9, wherein the elongated shaft has a thickness between 0.05 and .06 inches at the distal curved portion.

In Example 11, the system of any one of Examples 1-10, wherein the relief structures extend along a length between 1.2 and 1.3 inches of the distal curved portion.

In Example 12, the system of any one of Examples 1-11, wherein the distal curved portion comprises an alignment feature having a semi-circular profile positioned at a proximal end of the curved portion.

In Example 13, the system of Example 7, wherein the section proximal to the relief structures resists a predetermined threshold of bending.

In Example 14, the system of Example 13, wherein the section proximal to the relief structures has a length sufficient to provide mechanical impedance when the introducer cannula reaches an intended insertion depth.

In Example 15, the system of any one of Examples 1-14, wherein the treatment probe comprises a bipolar radiofrequency probe.

In Example 16, a system for treating a vertebral body is provided. The system comprises an introducer cannula having an introducer lumen disposed therethrough, a curved cannula having an elongated tube with a curved cannula lumen disposed therethrough, the elongated tube having a proximal straight portion and a distal curved portion, the elongated tube configured to be received into the introducer lumen of the introducer cannula; a stylet having an elongated shaft with a proximal straight portion and a distal curved portion, the elongated shaft configured to be received into the curved cannula lumen; wherein the distal curved portion of the stylet comprises relief structures along an outer curvature configured to maintain a consistent cross-sectional profile during deflection; and a treatment probe configured to be advanced through the curved cannula after removal of the stylet.

In Example 17, the system of Example 16, wherein the relief structures comprise a plurality of projections and recesses uniformly spaced along the outer curvature of the distal curved portion, wherein the plurality of projections and recesses are configured to create discrete flex points.

In Example 18, the system of Example 17, wherein the plurality of projections and recesses comprise a plurality of ridges radially-oriented and configured to engage with and support a curved cannula wall during deflection.

In Example 19, the system of Example 18, wherein each of the plurality of ridges has an edge profile to maintain smooth engagement with the curved cannula wall, wherein the edge profile is a filleted edge profile.

In Example 20, the system of Example 18, wherein each of the plurality of ridges are perpendicular to a longitudinal axis of the elongated shaft.

In Example 21, the system of Example 18, wherein the plurality of ridges have a depth configured to maintain structural integrity while enabling controlled bending.

In Example 22, the system of Example 16, wherein the distal curved portion includes a length proximal to the relief structures configured to prevent kinking.

In Example 23, the system of Example 16, wherein the stylet further comprises a distal channeling tip, wherein the distal channeling tip of the curved cannula has a sharper angle at the tip.

In Example 24, the system of Example 16, wherein the distal curved portion has a bend angle between 60 and 85degrees.

In Example 25, the system of Example 16, wherein the elongated shaft has a thickness between 0.05 and .06 inches at the distal curved portion.

In Example 26, the system of Example 16, wherein the relief structures extend along a length between 1.2 and 1.3 inches of the distal curved portion.

In Example 27, the system of Example 16, wherein the distal curved portion comprises an alignment feature having a semi-circular profile positioned at a proximal end of the curved portion.

In Example 28, the system of Example 16, wherein the treatment probe comprises a bipolar radiofrequency probe.

In Example 29, a medical device for accessing and navigating bone is provided. The device comprises a curved cannula comprising an elongated tube having a proximal straight portion, a distal curved portion, and a cannula lumen therethrough; a stylet comprising an elongated shaft comprising a proximal handle portion, a straight shaft portion, and a distal curved portion; wherein the distal curved portion comprises a plurality of ridges extending along an outer curvature; wherein the plurality of ridges have filleted edges to facilitate insertion and removal through the curved cannula.

In Example 30, the device of Example 29, wherein the plurality of ridges are uniformly spaced.

In Example 31, the device of Example 29, wherein the distal curved portion includes a length proximal to the plurality of ridges configured to prevent kinking during insertion.

In Example 32, the device of Example 29, wherein each of the plurality of ridges are perpendicular to a longitudinal axis of the elongated shaft.

In Example 33, the device of Example 29, wherein the distal curved portion has a bend angle of at least 75 degrees.

In Example 34, the device of Example 29, wherein the plurality of ridges maintain a horseshoe cross-section when subjected to lateral forces during bone penetration.

In Example 35, a method for accessing a target location within a vertebral body is provided. The method comprises inserting an introducer cannula through a pedicle adjacent to the vertebral body; advancing a curved cannula assembly through the introducer cannula, wherein the curved cannula assembly comprises a curved cannula and a stylet, wherein the stylet comprises and elongated shaft having a distal curved portion with a plurality of circumferential ridges along an outer radius of the distal curved portion; extending the curved cannula assembly beyond a distal end of the introducer cannula to create a curved path within the vertebral body, wherein the plurality of circumferential ridges maintain structural integrity during advancement through varying bone densities; removing the stylet from the curved cannula; and advancing a treatment device through the curved cannula to the target location.

For purposes of summarizing the disclosure, certain aspects, advantages, and novel features of embodiments of the disclosure have been described herein. It is to be understood that not necessarily all such advantages may be achieved in accordance with any particular embodiment of the disclosure provided herein. Thus, the embodiments disclosed herein may be embodied or carried out in a manner that achieves or optimizes one advantage or group of advantages as taught or suggested herein without necessarily achieving other advantages as may be taught or suggested herein.

The methods summarized above and set forth in further detail below describe certain actions taken by a practitioner; however, it should be understood that they can also include the instruction of those actions by another party. Thus, actions such as “applying thermal energy” include “instructing the applying of thermal energy.” Further aspects of embodiments of the disclosure will be discussed in the following portions of the specification. With respect to the drawings, elements from one figure may be combined with elements from the other figures.

### BRIEF DESCRIPTION OF THE DRAWINGS

Several embodiments of the disclosure will be more fully understood by reference to the following drawings which are for illustrative purposes only:

FIG. 1 illustrates various vertebral levels and vertebrae that may be treated by the systems and methods described herein.

FIG. 2 illustrates an example kit or system of access tools configured to access a vertebral body.

FIGS. 3A-3C include various views of an introducer cannula of the kit or system of FIG. 2.

FIG. 3D is a side view of an introducer stylet of the kit or system of FIGS. 2 and 3E is a side view of a distal cutting tip of an introducer stylet.

FIGS. 3F-3H illustrate a proximal portion of an introducer assembly of the kit or system of FIG. 2.

FIG. 3I is a side view and FIG. 3J is a top view of a curved cannula of the kit or system of FIG. 2.

FIG. 3K is a side view of a J-stylet of the kit or system of FIG. 2 and FIGS. 3L and 3M show a side view and a perspective view of a curved distal end portion of the J-stylet of FIG. 3K.

FIGS. 3N and 3O illustrate insertion of the J-stylet of FIGS. 3K-3M into the curved cannula of FIGS. 3I and 3J.

FIG. 3P illustrates insertion of the curved cannula assembly of the kit or system of FIG. 2 into the introducer cannula of FIGS. 3A-3C.

FIG. 3Q is a side cross-section view of a proximal portion of the introducer cannula and the curved distal end portions of the curved cannula assembly.

FIGS. 3R and 3S illustrate operation of a gear wheel of the curved cannula of FIGS. 3I and 3J in connection with insertion of the curved cannula assembly into the introducer cannula.

FIGS. 3T and 3U illustrate operation of a bail of the J-stylet of FIGS. 3K-3M to facilitate insertion and retraction of the J-stylet from the curved cannula.

FIG. 3V is a side view of a straight stylet of the kit or system of FIG. 2.

FIG. 3W is side cross-section view of a distal end portion of the straight stylet. FIG. 3WW is side cross-section view of an alternate embodiment of a distal end portion of the straight stylet.

FIGS. 3X and 3Y illustrate an optional introducer drill of the kit or system of FIG. 2.

FIG. 3Z illustrates the introducer drill inserted fully within the introducer cannula.

FIG. 4A illustrates a side view of a curved distal end portion with relief structures of a J-stylet.

FIG. 4B illustrates a detail side view of the curved distal end portion when straight of the J-stylet of FIG. 4A.

FIG. 4C illustrates a detail side view of a distal channeling tip of the J-stylet of FIG. 4A.

FIGS. 5A-5H illustrate various steps of a method of accessing and treating tissue within a vertebral body using one or more of the access tools of the kit or system of FIG. 2.

FIG. 6 illustrates an example radiofrequency generator.

### DETAILED DESCRIPTION

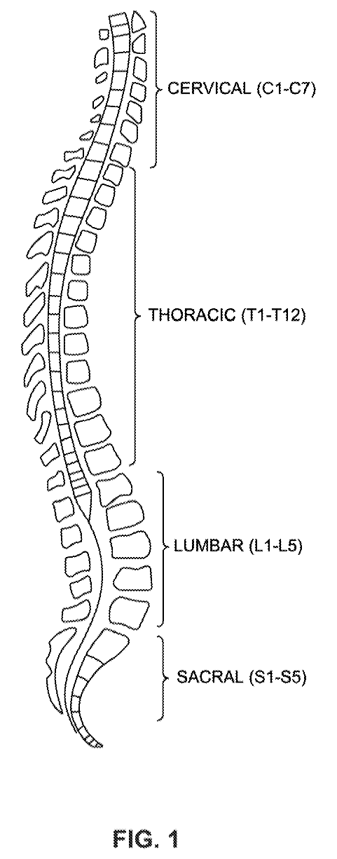

Several implementations described herein are directed to systems and methods for modulating nerves within or adjacent (e.g., surrounding) bone. In some implementations, an intraosseous nerve (e.g., basivertebral nerve) within a bone (e.g., vertebral body) of the spine is modulated for treatment, or prevention, of chronic back pain. The vertebral body may be located in any level of the vertebral column (e.g., cervical, thoracic, lumbar and/or sacral). FIG. 1 schematically illustrates a vertebral column and the various vertebral segments or levels. Multiple vertebral bodies may be treated in a single visit or procedure (simultaneously or sequentially). The multiple vertebral bodies may be located in a single spine segment (e.g., two adjacent vertebral bodies in the sacral spine segment (e.g., S1 and S2) or lumbar spine segment (e.g., L3, L4 and/or L5) or thoracic spine segment or cervical spine segment) or in different spine segments (e.g., an L5 vertebra in the lumbar spine segment and an S1 vertebra in the sacral spine segment). Intraosseous nerves within bones other than vertebral bodies may also be modulated. For example, nerves within a humerus, radius, femur, tibia, calcaneus, tarsal bones, hips, knees, and/or phalanges may be modulated.

In some implementations, the one or more nerves being modulated are extraosseous nerves located outside the vertebral body or other bone (e.g., at locations before the nerves enter into, or after they exit from, a foramen of the bone). Other tissue in addition to, or alternative to, nerves may also be treated or otherwise affected (e.g., tumors or other cancerous tissue or fractured bones). Portions of nerves within or on one or more vertebral endplates or intervertebral discs between adjacent vertebral bodies may be modulated.

The modulation of nerves or other tissues may be performed to treat one or more indications, including but not limited to chronic low back pain, upper back pain, acute back pain, joint pain, tumors in the bone, and/or bone fractures. The modulation of nerves may also be performed in conjunction with bone fusion or arthrodesis procedures so as to provide synergistic effects or complete all-in-one, “one-and-done” treatment that will not require further surgical or minimally invasive interventions.

In some implementations, fractures within the bone may be treated in addition to denervation treatment and/or ablation of tumors by applying heat or energy and/or delivering agents or bone filler material to the bone. For example, bone morphogenetic proteins and/or bone cement may be delivered in conjunction with vertebroplasty or other procedures to treat fractures or promote bone growth or bone healing. In some implementations, energy is applied and then agents and/or bone filler material is delivered in a combined procedure. In some aspects, vertebral compression fractures (which may be caused by osteoporosis or cancer) are treated in conjunction with energy delivery to modulate nerves and/or cancerous tissue to treat back pain.

In accordance with several implementations, the systems and methods of treating back pain or facilitating neuromodulation of intraosseous nerves described herein can be performed without surgical resection, without general anesthesia, without cooling (e.g., without cooling fluid), and/or with virtually no blood loss. In some embodiments, the systems and methods of treating back pain or facilitating neuromodulation of intraosseous nerves described herein facilitate easy retreatment if necessary. In accordance with several implementations, successful treatment can be performed in challenging or difficult-to-access locations and access can be varied depending on bone structure or differing bone anatomy. One or more of these advantages also apply to treatment of tissue outside of the spine (e.g., other orthopedic applications or other tissue).

### ACCESS TO THE VERTEBRAL BODY

### Methods of Access

Various methods of access may be used to access a vertebral body or other bone. In some implementations, the vertebral body is accessed transpedicularly (through one or both pedicles). In other implementations, the vertebral body is accessed extrapedicularly or parapedicularly (e.g., without traversing through a pedicle or traversing adjacent to a pedicle). In some implementations, the vertebral body is accessed using an extreme lateral approach or a transforaminal approach, such as used in XLIF and TLIF interbody fusion procedures. In some implementations, an anterior approach is used to access the vertebral body.

In some implementations, the vertebral body may be accessed transforaminally through a basivertebral foramen. Transforaminal access via the spinal canal may involve insertion of a “nerve finder” or nerve locator device and/or imaging/diagnostic tool to avoid damaging spinal cord nerves upon entry by the access tools or treatment devices. The nerve locator device may comprise a hand-held stimulation system such as the Checkpoint Stimulator and Locator provided by Checkpoint Surgical^{®} or the EZstim^{®} peripheral nerve stimulator/nerve locators provided by Avanos Medical, Inc. The nerve finder or nerve locator device could advantageously identify sensitive nerves that should be avoided by the access tools so as not to risk paralysis or spinal cord injury upon accessing the target treatment site. The nerve locator device may be configured to apply stimulation signals between two points or locations and then assess response to determine presence of nerves in the area between the two points or locations. The nerve locator device may include a bipolar pair of stimulation electrodes or monopolar electrodes. In some implementations, the nerve locator features may be implemented on the access tools or treatment devices themselves as opposed to a separate stand-alone device.

### Access Tools

Access tools may include an introducer assembly including an outer cannula and a sharpened stylet, an inner cannula configured to be introduced through the outer cannula, and/or one or more additional stylets, curettes, or drills to facilitate access to an intraosseous location within a vertebral body or other bone. The access tools (e.g., outer cannula, inner cannula, stylets, curettes, drills) may have pre-curved distal end portions or may be actively steerable or curveable. Any of the access tools may have beveled or otherwise sharp tips or they may have blunt or rounded, atraumatic distal tips. Curved drills may be used to facilitate formation of curved access paths within bone. Any of the access tools may be advanced over a guidewire in some implementations. In some implementations, the stylet may comprise relief structures along the outer curvature of its distal curved portion. The relief structures comprise uniformly spaced projections and recesses with radially-oriented ridges. These ridges may be configured to maintain consistent cross-sectional profiles during deflection, support curved cannula walls during navigation, enable smooth advancement and retraction, and preserve structural integrity across varying bone densities.

In some implementations, an outer cannula assembly (e.g., introducer assembly) includes a straight outer cannula and a straight stylet configured to be received within the outer cannula. The outer cannula assembly may be inserted first to penetrate an outer cortical shell of a bone and provide a conduit for further access tools to the inner cancellous bone. An inner cannula assembly may include a cannula having a pre-curved or steerable distal end portion and a stylet having a corresponding pre-curved or steerable distal end portion. Multiple stylets having distal end portions with different curvatures may be provided in a kit and selected from by a clinician. The inner cannula assembly may alternatively be configured to remain straight and non-curved.

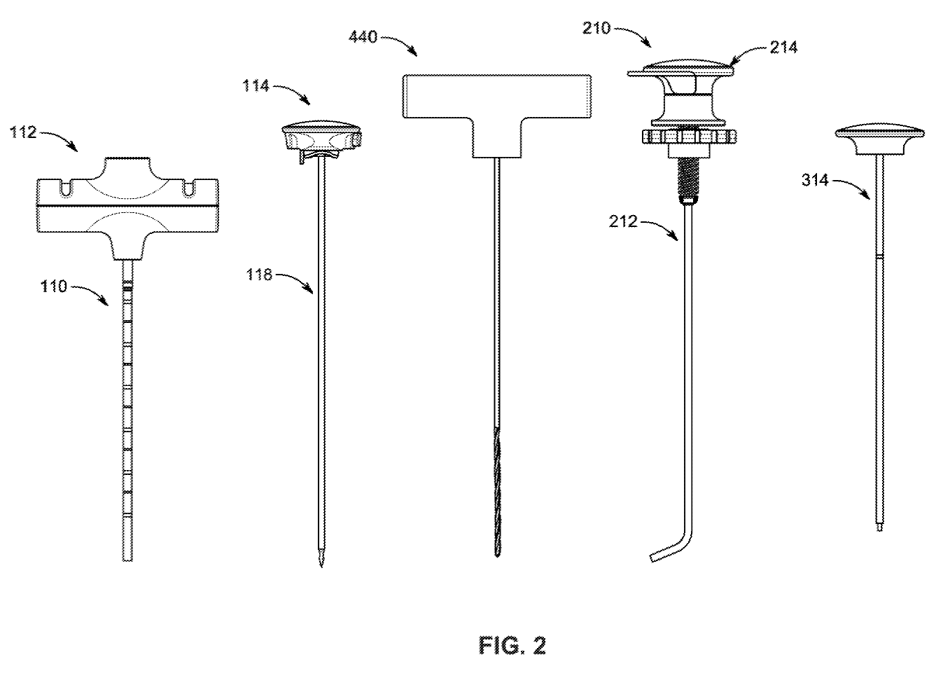

With reference to FIG. 2, in one implementation, a kit or system of access tools includes an introducer assembly 110 comprised of an introducer cannula 112 and an introducer stylet 114, a curved cannula assembly 210 comprised of a curved cannula 212 and a J-stylet 214, and a straight stylet 314. The introducer stylet 114 may be bevel tipped, trocar tipped, and/or diamond tipped. The introducer stylet 114 is configured to be received in a lumen of the introducer cannula 112 in a manner such that a distal tip of the introducer stylet 114 protrudes from an open distal tip of the introducer cannula 112, thereby forming the introducer assembly 110 in combination. The J-stylet 214 is configured to be received in a lumen of the curved cannula 212 in a manner such that a distal tip of the J-stylet 214 protrudes from an open distal tip of the curved cannula 212, thereby forming the curved cannula assembly 210 in combination. The curved cannula 212 and the J-stylet 214 may each comprise a straight proximal main body portion and a curved distal end portion. The curves of the curved distal end portions of the curved cannula 212 and the J-stylet 214 may correspond to each other. The straight stylet 314 is a flexible channeling stylet configured to be delivered through the curved cannula 212 and then to form and maintain a straight or generally straight path upon exiting the open distal tip of the curved cannula 212.

The access tools of FIG. 2 may be formed of a variety of rigid and flexible materials. The straight hypotube portion of the introducer cannula 112 may be made of a hard and stiff material, e.g., metal, hard plastic, ceramic, or composite material. The proximal handle portion of the introducer cannula 112 at the proximal end may be made of a plastic material or another type of suitable material that is capable of withstanding impact from a mallet. The straight rod or shaft portion of the introducer stylet 114 may be made of a hard and stiff material, e.g., metal, hard plastic or ceramic, and the proximal handle portion of the introducer stylet 112 may be made of a plastic material or a material that is capable of withstanding impact. The same materials can be applied to the different portions of the curved cannula assembly 210, including the curved cannula 212 and the J-stylet 214, and the straight stylet 314.

However the curved distal end portions of the curved cannula 212 and the J-stylet 214 and at least the distal end portion of the straight stylet 314 may be made of flexible and resilient materials. The flexibility and resilience of an access tool may depend on its dimensions, especially the cross-sectional dimensions, and the material it is made of. In some embodiments these flexible and resilient portions of the access tools can be made of one or more plastic materials, e.g., polyamide (PA), polyethylene terephthalate (PET), polycarbonate (PC), polyoxymethylene (POM), acrylonitrile butadiene styrene (ABS), polyethylene (PE), polypropylene (PP), and polyimide (PI). In some embodiment, the flexible and resilient portions can be made of certain metallic alloys, e.g., nitinol. In some embodiments, when the cross-sectional dimensions are relatively thick, they can also be made of one or more of the hard plastic elastomers, e.g., polyethylene-based polyolefin elastomers, polypropylene-based elastomers, thermoplastic polyester elastomers, thermoplastic polyurethane elastomers, nitrile butadiene rubber, and thermoplastic vulcanizate polymers. For example, the distal portion of the straight rod 156 can be a solid rod made of a plastic elastomer having a high Shore durometer measurement. In some embodiments, if the resilient tool portions have small cross-sectional dimensions or tubular structure to facilitate bending, they can be made of a semi-rigid materials, because when cross-sectional dimensions are sufficiently small proper flexibility can be achieved using semi-rigid materials. Further, chevron designs or patterns, or through-thickness slits or holes can be applied to make a rigid portion more flexible.

The access tools may be provided as a kit that may include one or more additional introducer cannulas 112, one or more additional introducer stylets 114 (e.g., with different tips, such as one with a bevel tip and one with a diamond or trocar tip), one or more additional curved cannulas 212 (e.g., having a curved distal end portion of a different curvature than a first curved cannula), an one or more additional J-stylets 214 (e.g., having a different curvature or different design configured to access different hardness of bone), an introducer drill 440, and/or an additional straight stylet 314 (e.g., having a different length from the first straight stylet). Some kits may include add-on components or accessory kit modules for accessing hard bone (e.g., the introducer drill 440 and J-stylet 214 specially configured to access hard bone). Some kits may include additional access tool components or accessory kit modules adapted to access one or more additional vertebrae in the same spinal segment or in different spinal segments. The kit may also include one or more (e.g., at least two) treatment devices (such as radiofrequency energy delivery probes).

In some embodiments, the access tools (e.g., kit) may be specifically designed and adapted to facilitate access to hard, non-osteoporotic bone (e.g., bone surrounding or within a vertebral body, such as a cervical vertebra, a thoracic vertebra, a lumbar vertebra, or a sacral vertebra). Hard bone may be determined based on bone mass density testing, compressive strength determinations, compressive modulus determinations, imaging modalities, or based on tactile feel by an operator as access instruments are being advanced. In some implementations, hard bone may be determined as bone having a bone mineral density score within a standard deviation of a normal healthy young adult (e.g., a T score greater than or equal to -1). In some implementations, hard bone may be identified as bone having a compressive strength of greater than 4 MPa and/or a compressive modulus of greater than 80 MPa for cancellous bone. For cortical bone, the compressive strength may be greater than 5.5 MPa and a compressive modulus may be greater than 170 MPa.

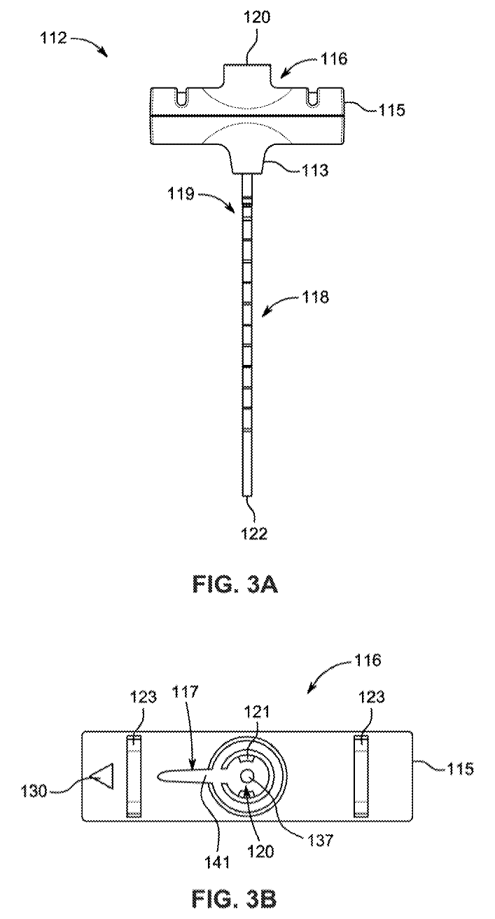

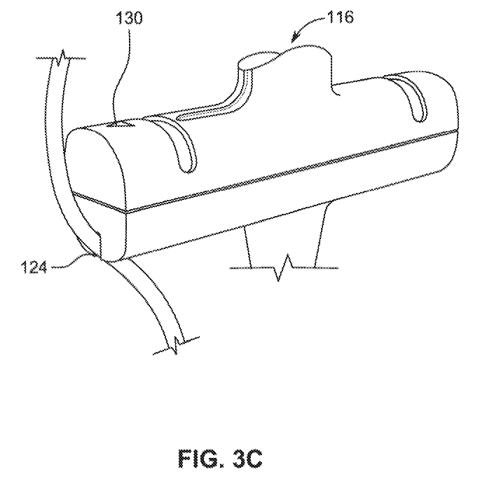

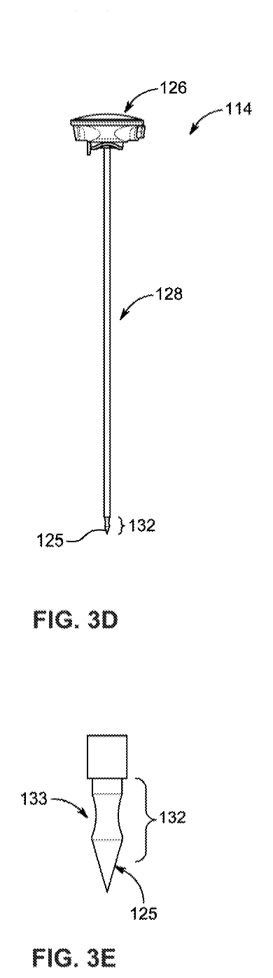

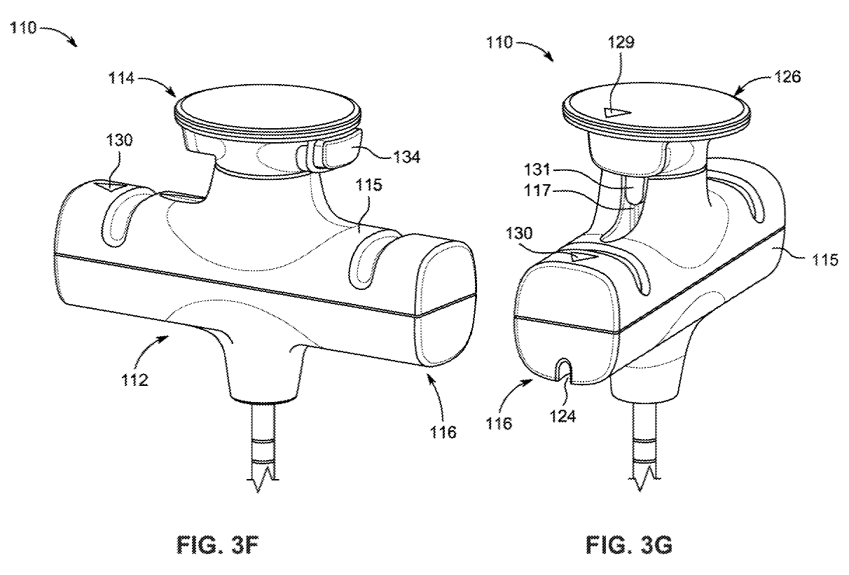

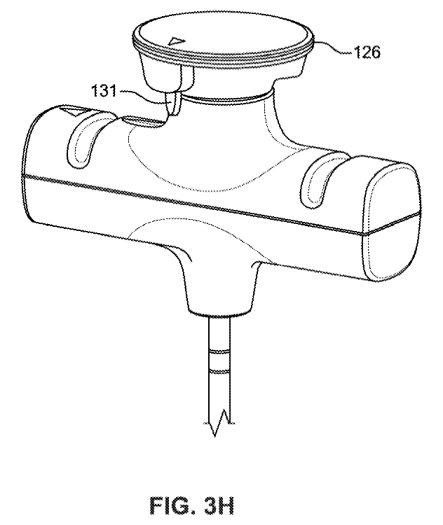

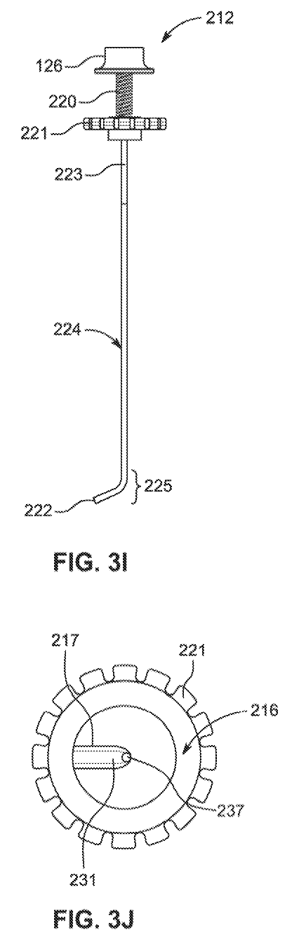

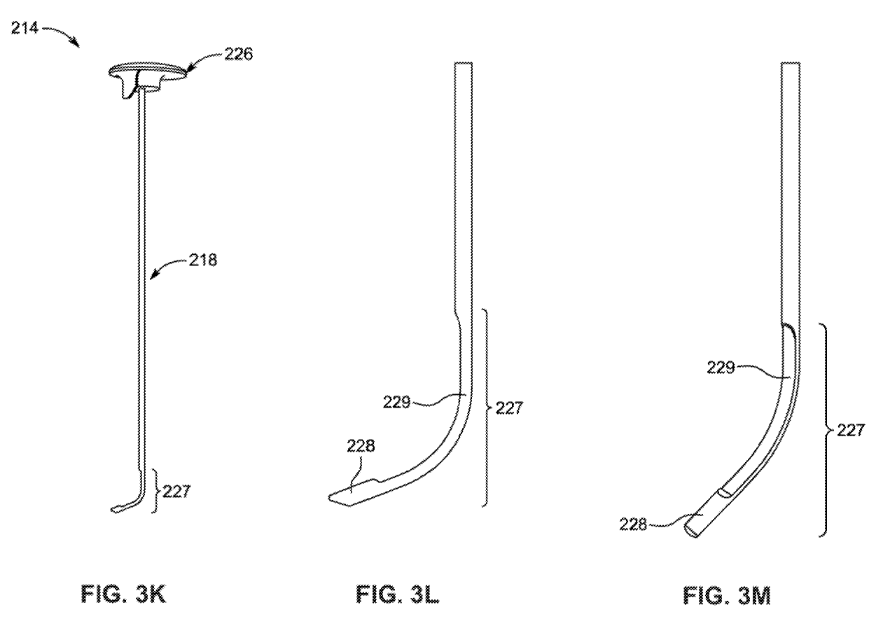

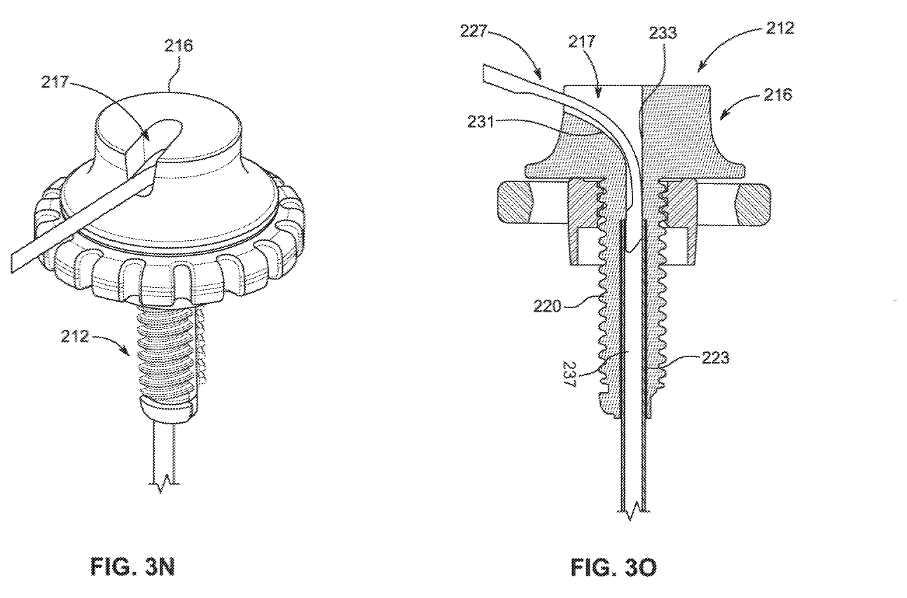

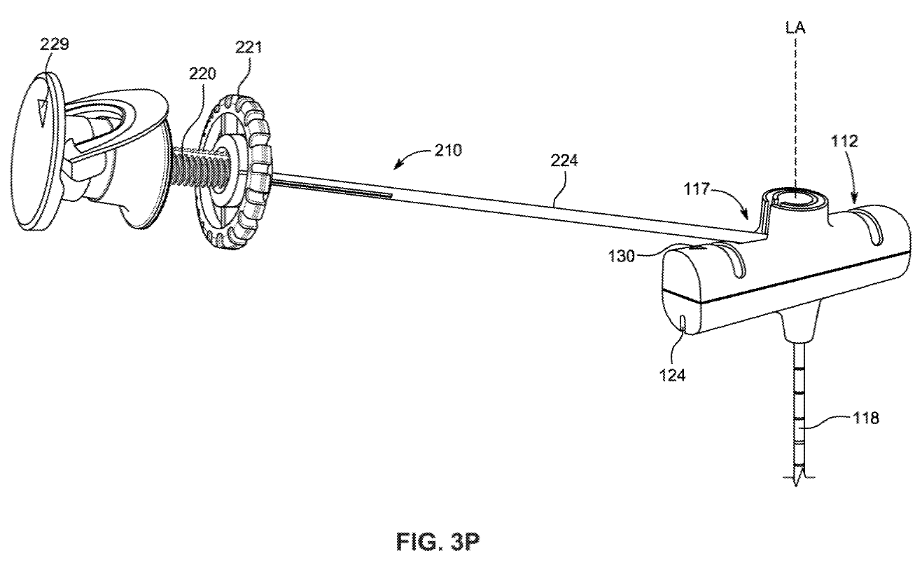

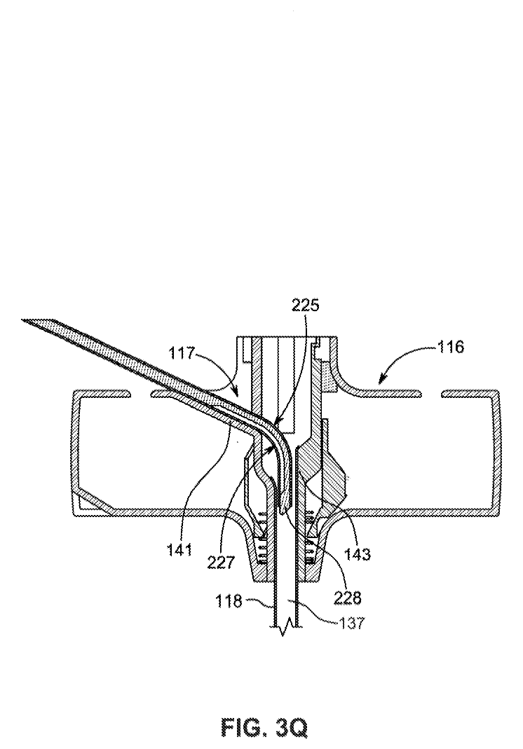

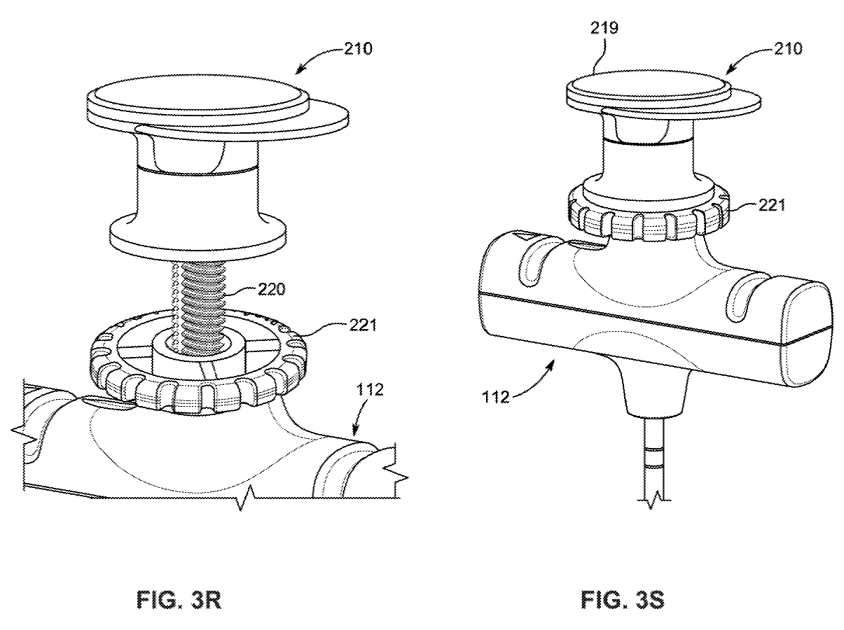

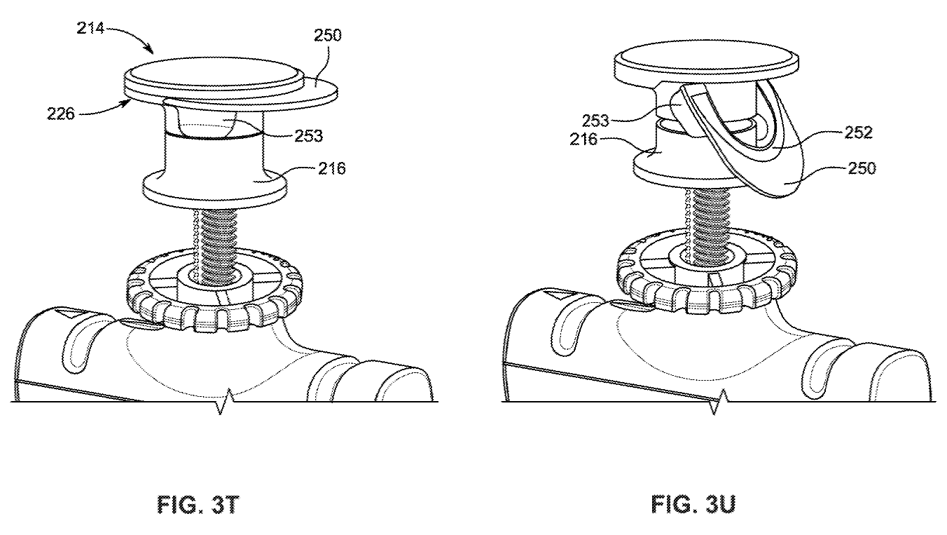

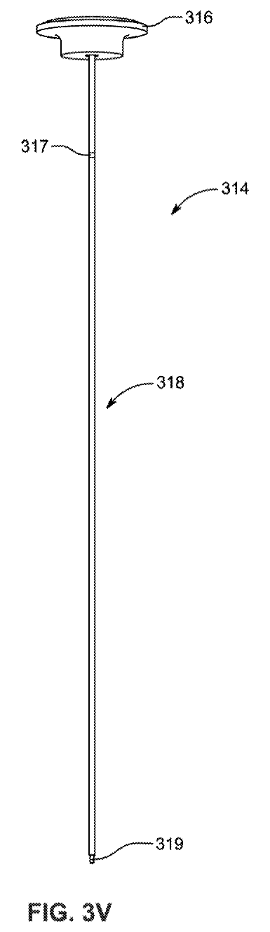

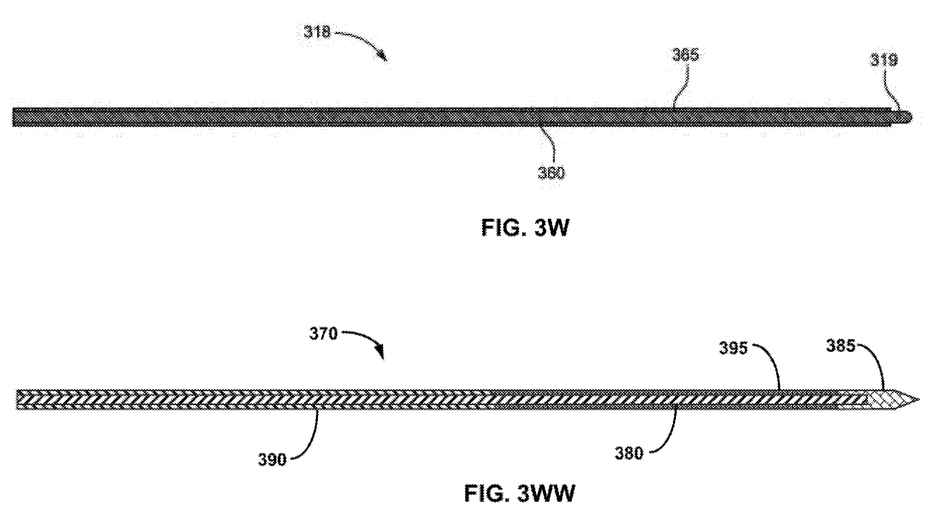

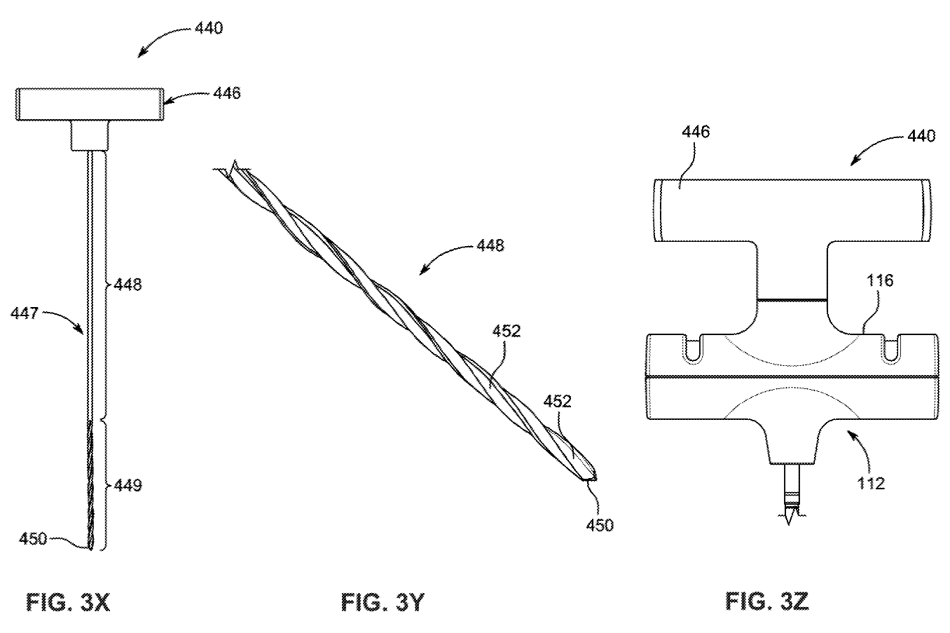

FIGS. 3A-3C illustrate various views of an embodiment of the introducer cannula 112. The introducer cannula 112 includes a proximal handle 116 and a distal hypotube 118 extending from the proximal handle 116. The illustrated proximal handle 116 comprises a “smokestack” or “T-Handle” design configuration adapted to provide sufficient finger clearance and gripping (e.g., one finger on each side of a lower flange 113 of the proximal handle 116 and along the lower surface of a crossbar portion 115) to facilitate removal. However, alternative design configurations for the proximal handle other than a “smokestack” or “T-handle” design may be incorporated.

The proximal handle 116 includes an upper central opening 120 configured to facilitate straight axial insertion of an introducer stylet 114 or other straight access tool. The upper central opening 120 may be positioned so as to correspond with (e.g., be coaxial with) a central lumen 137 extending through the hypotube 118 of the introducer cannula 112 so as to facilitate insertion of straight instruments (e.g., the introducer stylet 114 or steerable cannulas or steerable stylets) therethrough. The proximal handle 116 may also include coupling features 121 (e.g., recesses, notches, grooves, tabs) to facilitate coupling or mating of a proximal handle 216 of the introducer stylet 114 with the proximal handle 116 of the introducer cannula 112. The coupling features 121 may be adapted to prevent rotation of the introducer stylet 114 and/or to provide assurance that a distal tip 125 of the introducer stylet 114 extends beyond an open distal tip 122 of the hypotube 118 of the introducer cannula 112. As such, penetration of the distal tip 125 of the introducer stylet 114 is facilitated through bone. The upper surface of the proximal handle 116 of the introducer cannula 112 also includes a curved lateral slot 117 and curved ramp 141 to facilitate insertion of the curved cannula assembly 210 into the proximal handle 116 and then into and along the central lumen of the hypotube 118, as will be further described subsequently.

The central lumen 137 of the hypotube 118 extends from the proximal handle 116 to the open distal tip 122 of the hypotube 118. The hypotube 118 may be flared or tapered such that the diameter of the hypotube 118 is not constant along its entire length. For example, the diameter may decrease abruptly at a certain distance (e.g., 1 cm – 3 cm) from a lower edge of the lower flange 113 of the proximal handle 116, forming an abrupt narrowing edge 119, and then continue with a constant diameter distally. In another embodiment, the diameter may decrease gradually (e.g., taper uniformly) along the length of the hypotube 118 from the lower edge of the lower flange 113 or the edge 119 to the open distal tip 122 of the hypotube 118. The central lumen 137 of the hypotube 118 may be coated with a medical grade silicone lubricant to improve tool insertion and removal. The outer diameter of the hypotube 118 may range from about 4.2 mm to about 4.5 mm, e.g., from 3 mm to 5 mm. As shown in FIG. 3C, a lower (bottom) side surface of the proximal handle 116 of the introducer cannula 112 may include a cutout 124 adapted to receive a portion of a flexible shaft of a treatment device (e.g., radiofrequency probe comprised of nitinol, cable assembly of the radiofrequency probe, or other flexible or shape memory material) and hold it in place and out of the way during a treatment procedure, thereby reducing stack height (e.g., by approximately 3 inches /75 mm or more). In some embodiments there maybe more than one cutout 124.

FIGS. 3D-3H illustrate various views and portions of embodiments of introducer stylets 114. FIG. 3D illustrates a side view of an introducer stylet 114. The introducer stylet 114 includes a proximal handle 126 and a distal elongate member or shaft 128. The proximal handle 126 comprises an upper surface that is adapted for malleting by a mallet and a lower surface that is adapted to facilitate removal of the introducer stylet 114 by an operator. The length of the distal elongate member 128 may range from 125 mm to 300 mm (e.g., 125 mm to 200 mm, 175 mm to 250 mm, 200 mm to 225 mm, 215 mm to 300 mm). The distal end portion 132 of the introducer stylet 114 may comprise a scalloped section 133 (as shown more closely in FIG. 3E) to provide a release mechanism for bone compaction. The scalloped section 133 may be designed to have a side profile shaped generally like an hourglass. The scalloped section 133 may gradually taper down from a full diameter proximal portion to a narrow-most middle potion and then gradually taper back to a full diameter distal portion. The taper may be symmetric or asymmetric. The scalloped section 133 may comprise one scallop (or scooped-out region) or multiple scallops (or scooped-out regions) along the length of the distal end portion 132. A distal tip 125 of the distal end portion 132 may comprise a full diameter so as to be adapted to break apart bone (e.g., pedicle bone, cortical bone of a vertebral body). As the bone is broken up by the distal tip 125 of the distal end portion 132, bone shards or chips can pack into a gap formed between the distal end portion 132 of the introducer stylet 114 and the inner surface of the distal end portion of the introducer cannula 112, thereby making it more difficult for the introducer stylet 114 to be removed from the introducer cannula 112. In accordance with several embodiments, the scalloped section 133 of the introducer stylet 114 advantageously provides the bone shards and fragments a place to fall into during removal of the introducer stylet 114 so as to facilitate easier removal of the introducer stylet 114.

FIGS. 3F-3H illustrate the introducer assembly 110 after the introducer stylet 114 has been inserted into the introducer cannula 112. As indicated above, the proximal handle 116 of the introducer cannula 112 may include mating or engagement features (e.g., coupling features 121) that facilitate automatic engagement of the introducer stylet 114 with the proximal handle 116 of the introducer cannula 112. In some embodiments the automatic engagement may be a snap-fit.

The proximal handle 126 of the introducer stylet 114 includes an alignment indicator 129, an anti-rotation tab 131, and a press button 134. As shown best in FIG. 3G, the alignment indicator 129 is configured to align with a corresponding alignment indicator 130 on the upper surface of the crossbar portion 115 of the proximal handle 116 of the introducer cannula 112 in order to ensure proper insertion and alignment of the introducer stylet 114 with respect to the introducer cannula 112. The anti-rotation tab 131 is configured to be positioned within the slot 117 of the proximal handle 116 of the introducer cannula 112 and to prevent rotation of the introducer stylet 114 with respect to the introducer cannula 112 during malleting and orienting.

The press button 134 is integrally coupled to the anti-rotation tab 131 such that pressing of the press button 134 extends the anti-rotation tab 131 out of the constraint of the slot 117, thereby allowing the introducer stylet 114 to rotate with respect to the introducer cannula 112 (as shown in FIG. 3H). Pressing the press button 134 also releases engagement of the introducer stylet 114 with the introducer cannula 112 to enable removal of the introducer stylet 114 from the introducer cannula 112. The proximal handle 126 of the introducer stylet 114 may include internal ramps (not shown) configured to provide a mechanical advantage to assist in removal of the introducer stylet 114 from the introducer cannula 112 (especially if bone shards have packed into gaps between the introducer stylet 114 and introducer cannula 112 making removal more difficult) as the proximal handle 126 is rotated (e.g., 120-degree rotation counter-clockwise). The combination of the scalloped distal end portion design and the internal ramps in the proximal handle 126 may provide increased reduction of removal forces by 50% – 70% compared to a full diameter (e.g., no scalloped section) distal end portion design with no ramps in the proximal handle 126.

FIGS. 3I and 3J illustrate a side view and a top view of an embodiment of the curved cannula 212. The curved cannula 212 includes a proximal handle 126, a threaded proximal shaft portion 220, a gear wheel 221, a rigid support portion 223, and a distal flexible shaft portion 224. The proximal handle 216 includes a curved slot 217 and a curved ramp 231 configured to facilitate insertion of the J-stylet 214 into and along a central lumen 237 of the curved cannula 212 extending from the proximal handle 216 to an open distal tip 222 of the distal flexible shaft portion 224. The central lumen 237 of the curved cannula 212 may be coated with a medical grade silicone lubricant to improve tool insertion and removal. In various embodiments, the curved cannula 212 does not include a rigid support portion 223.

In the illustrated example, the gear wheel 221 comprises internal threads configured to interface with corresponding external threads of the threaded proximal shaft portion 220 such that rotation of the gear wheel 221 causes controlled proximal or distal translation of the gear wheel 221 along the threaded proximal shaft portion 220. The threaded proximal shaft portion 220 is sized such that when the gear wheel 221 is in its distal-most position, the distal tip 222 of the curved cannula 212 does not extend out of the open distal tip 122 of the introducer cannula 112 when the curved cannula assembly 210 is fully inserted therein. The gear wheel 221 may spin freely about the threaded proximal shaft portion 220. The threads may comprise triple threads. The gear wheel 221 may be configured to traverse the entire length of the threaded proximal shaft portion 220 with four complete rotations of the gear wheel 221. In some embodiments, when in use, the distal tip 222 does stick out of the distal opening 122.

The rigid support portion 223 may comprise a biocompatible metal or other rigid material, such as stainless steel, titanium, platinum and/or the like, so as to provide additional support to the curved cannula 212 during insertion of the J-stylet 214. The distal flexible shaft portion 224 may be comprised of a thermoplastic, shape-memory polymer material (such as polyether ether ketone (PEEK), polyurethane, polyethylene terephthalate (PET), and/or the like). The distal end portion 225 may be pre-curved (e.g., shape-set) to have a predetermined curve in a “resting” or unconstrained configuration.

FIGS. 3K-3M illustrate an embodiment of the J-stylet 214. FIG. 3K illustrates a side view of the J-stylet 214 in a “resting” normal, unconstrained configuration or state and FIGS. 3L and 3M are close-up views (side view and perspective view, respectively) of a curved distal end portion 227 of the J-stylet 214. The J-stylet 214 comprises a proximal handle 226 and a distal elongate shaft 218. The proximal handle 226 comprises an upper surface that is adapted for malleting by a mallet and a lower surface that is adapted to facilitate removal of the J-stylet 214 by two or more (e.g., two, three, or four) fingers of an operator. The upper surface of the proximal handle 226 includes an alignment indicator 229 (shown, for example, in FIG. 3P) configured to be aligned with the corresponding alignment indicator 130 of the introducer cannula 112 to facilitate insertion, removal, and deployment of the J-stylet 214 (and curved cannula assembly 210).

The distal elongate shaft 218 includes a curved distal end portion 227 having an asymmetric curve profile along its length (e.g., the curved distal end portion 227 does not have a constant full diameter along its length). A distal channeling tip 228 is sized and shaped to facilitate channeling through cancellous bone along a curved path or trajectory. The curved distal end portion 227 comprises a flexible curved section 229 having a “D-shaped” cross-sectional profile, as shown, for example, by the cross-sectional profile. A cross-sectional view of the “D-shaped” cross-sectional profile of the curved section 229 may be a partial circle. The curved section 229 may be formed by mechanical grinding or cutting of a cylindrical wire, before the curvature is formed, until the desired D-shaped cross section profile is achieved in which a top (e.g., inner) surface of the curved section 229 is generally smooth and flat. The thickness (e.g., vertical cross-sectional dimension) of the curved section 229, the predefined set angulation or radius of curvature, and the starting and ending points of the flexible curved section 229 along the length of the curved distal end portion 227 may be varied to provide J-stylets having different rigidity and bending characteristics for different levels of vertebrae or different densities of bone.

According to several embodiments, the asymmetric curve profile (e.g., profile with D-shaped cross-section) advantageously provides improved cephalad-caudal steering because the curved distal end portion 227 primarily bends inward and not laterally. In addition, the design and material of the curved distal end portion 227 of the J-stylet 214 may enable the angle of curvature of the curved distal end portion 227 to advantageously remain relatively consistent and reproducible across a variety of bone densities, or regardless of bone environment. For example, in one embodiment, the design and material of the curved distal end portion 227 of the J-stylet 214 facilitates consistent and reproducible access to a posterior location (e.g., in a posterior half of the vertebral body or to a location approximately 30%-50% of the distance between the posterior-most aspect and the anterior-most aspect of the vertebral body along a sagittal axis or to a geometric center or midpoint within the vertebral body for vertebral bodies having varying bone densities or other desired target location in the vertebral body or other bone). In accordance with several embodiments, the curvature is designed to deviate by less than 35 degrees (e.g., less than 30 degrees, less than 25 degrees, less than 20 degrees, less than 15 degrees, less than 10 degrees) or less than 30% from the predefined set curvature of the curved distal end portion 227 in an unconstrained configuration (even in hard bone, such as non-osteopenic or non-osteoporotic bone).

Various embodiments of the curved sections 229 may exist for improved performance during an operation and/or for easier removal of the J-stylet 214 from the curved cannula 212, as will be further described subsequently.

The J-stylet 214 may be designed and adapted to exert a lateral force of between 6 pounds and 12 pounds. The curvature of the curved section 229 of the curved distal end portion 227 may result in an angle between the central longitudinal axis of the straight proximal portion of the distal elongate shaft 218 and an axis of the distal channeling tip 228 in a normally unconstrained state. This angle may be between 65 degrees and 80 degrees (e.g., 65 degrees, 70 degrees, 75 degrees, 80 degrees, or any other value within the recited range). The radius of curvature of the curved section 229 may range from 11.5 mm to 15 mm (e.g., from 11.5 mm to 12 mm, from 12 mm to 12.5 mm, from 12 mm to 13 mm, from 12.5 mm to 14 mm, from 13 mm to 14 mm, from 13.5 mm to 15 mm, overlapping ranges thereof, or any value within the recited ranges). The J-stylet 214 may comprise of nitinol, other flexible metallic alloy material, or plastic material.

FIGS. 3N and 3O are a perspective view and a side cross-sectional view, respectively, illustrating insertion of the curved distal end portion 227 of the J-stylet 214 into the slot 217 of the proximal handle 216 of the curved cannula 212. As shown in FIG. 3O, the slot 217 comprises a curved ramp 231 and a straight vertical backstop support 233 (e.g., with no trumpeted section) to facilitate insertion of the curved distal end portion 227 of the J-stylet 214. As indicated above, the curved cannula 212 includes the rigid support portion 223 extending into and out of the threaded shaft portion 220 to provide additional support upon insertion of the J-stylet 214 within the central lumen 237 of the curved cannula 212.

FIGS. 3P and 3Q illustrate insertion of the curved cannula assembly 210 into the introducer cannula 112. The curved distal end portion 225 of the curved cannula assembly 210 is inserted into the slot 117 in the proximal handle 116 from a side angle. The side angle may be about 65 to 75 degrees with respect to the central longitudinal axis LA of the distal hypotube 118 of the introducer cannula 112, e.g., with an about 70 degree starting angle in one embodiment. During insertion, the curved distal end portion 225 may follow along the ramp 141 in the slot 117 and down the central lumen 137 of the distal hypotube 118 of the introducer cannula 112. The gear wheel 221 of the curved cannula 212 may be in a distal-most position along the threaded proximal portion 220 of the curved cannula 212 during the insertion. As such inadvertent advancement of the curved distal portion 225 of the curved cannula assembly 210 beyond the open distal tip 122 of the introducer cannula 112 may be prevented until the operator is ready to further advance. In some embodiments, the advancement of the curved distal portion 225 of the curved cannula assembly 210 may extend beyond the distal open tip 122 when in use.

Referring to FIG. 3Q, a close-up side cross-section view of the proximal portion of the introducer cannula 112 and the curved distal portion 225 of the curved cannula assembly 210 is illustrated. As shown, the introducer cannula 112 is shaped so as to provide a backstop support 143 generally aligned with the inner surface of the central lumen 137 of the hypotube 118. As such, the curved distal end portion 225 of the distal flexible shaft portion 224 of the curved cannula 212 may not pivot out of the introducer cannula 112 upon insertion. In accordance with several embodiments, the asymmetric “D-shaped” cross-sectional profile of the J-stylet 214 may be advantageously designed to prevent twisting during insertion.

FIGS. 3R and 3S illustrate operation of the gear wheel 221 of the curved cannula 212. As shown in FIG. 3R, the gear wheel 221 is rotated until it is in its distal-most position along the threaded proximal portion 220 prior to the insertion of the curved cannula assembly 210 within the introducer cannula 112 to prevent inadvertent advancement of the curved distal end portion 225 of the curved cannula assembly 210. out of the introducer cannula 112. In some embodiments, the advancement of the curved distal portion 225 of the curved cannula assembly 210 may extend beyond out of the introducer cannula 112.

As shown in FIG. 3S, the gear wheel 221 is rotated to its proximal-most position along the threaded proximal portion 220 to enable full insertion of the curved cannula assembly 210 within the introducer cannula 112. As such, the curved distal end portion 225 of the curved cannula assembly 210 may extend out of the introducer cannula 112 and along a curved path within the cancellous bone region of the vertebral body or other bone.

FIGS. 3T and 3U illustrate operation of an actuation mechanism of the J-stylet 214. The proximal handle 226 of the J-stylet 214 includes an actuator 250 configured to be toggled between a first “resting” or “inactive” configuration and a second “active” configuration. In the first, or “inactive”, configuration the actuator 250 is generally aligned with (e.g., parallel or substantially parallel to) the upper surface of the proximal handle 226, as shown in FIG. 3T. In the second, or “active”, configuration the actuator 250 is offset from the upper surface of the proximal handle 226, as shown in FIG. 3U. The actuator 250 is configured to act as a lever to cause a slight axial, e.g., proximal-distal, movement of the J-stylet 214 with respect to the curved cannula 212 as the actuator 250 is pivoted. When the actuator 250 is toggled to the “active” configuration, flanges 253 of the actuator 250 contacts the proximal handle 216 of the curved cannula 212 to cause proximal retraction of the J-stylet 214 with respect to the curved cannula 212. As such, the distal channeling tip 228 of the J-stylet 214 resides within the curved cannula 212 and does not extend out of the open distal tip 222 of the curved cannula 212. In some embodiments, J-stylet 214 resides completely within the curved cannula 212 and does not extend out of the open distal tip 222 of the curved cannula 212. In accordance with several embodiments, the actuator 250 is advantageously toggled to the “active” configuration (in which the distal channeling tip 228 of the J-stylet 214 resides within the open distal tip 222 of the curved cannulas 212) upon insertion or removal of the curved cannula assembly 210 from the introducer cannula 112, or upon insertion or removal of the J-stylet 214 from the curved cannula 212 (e.g., so as to avoid friction caused by interaction between two metal components). In some embodiments, it may be advantageous to be in an active position when the curved cannula assembly 210 is inserted into the introducer cannula 112 to avoid metal-to-metal friction. It may also be advantageous to be in the active position when removing from the curved cannula 212 when the curved cannula is still inserted in the introducer cannula 112 to help start removal by bringing the J-Stylet 214 tip into a distal exit of the curved cannula 212. The upper surface of the actuator 250 may include an indicator 252 (e.g., colored marking or other visual indicator) that is visible to an operator when the actuator 250 is in the active configuration and invisible when the actuator 250 is in the inactive configuration.

FIG. 3V illustrates a slight side perspective view of an embodiment of the straight stylet 314. FIG. 3W illustrates a distal portion of the straight stylet 314. The straight stylet 314 includes a proximal handle 316 and a distal elongate shaft 318. The proximal handle 316 includes an upper surface adapted for malleting by a mallet or application of pressure by a hand or fingers of an operator. A marker band 317 may be positioned along the distal elongate shaft 318 at a position corresponding to the position when a distal channeling tip 319 of the straight stylet 314 is exiting the open distal tip 222 of the curved cannula 212 as the straight stylet 314 is advanced through the curved cannula 212. The marker band 317 can be a visual marking on the distal elongate shaft 318 and/or the marker band 317. The length of the straight stylet 314 may be sized such that, when the straight stylet 314 is fully inserted within the curved cannula 212, the length of the shaft portion of the straight stylet 314 extending beyond the open distal tip 222 of the curved cannula 212 is between 25 and 50 mm (e.g., between 25 mm and 35 mm, between 30 mm and 40 mm, between 35 mm and 45 mm, between 40 and 50 mm, overlapping ranges thereof, or any value within the recited ranges). The diameter of the straight stylet 314 may be sized so as to be inserted within and through the central lumen 237 of the curved cannula 212 with minimal frictional resistance. The distal elongate shaft 318 may comprise an inner flexible, shape memory core 360 extending from the proximal handle 316 to the distal channeling tip 319 of the straight stylet 314 and a flexible (e.g., polymeric) outer layer 365 extending from the proximal handle 316 to a distal end of the distal elongate shaft 318 but stopping short (or proximal to) the distal channeling tip 319. As such the inner core 360 protrudes out of the outer layer 365. The straight stylet 314 may be flexible enough to bend to traverse the curved distal end portion 225 of the curved cannula 212 without significant friction. The straight stylet 314 may also be sufficiently rigid so as to maintain a straight path once the straight stylet 314 exits the open distal tip of the curved cannula 212. The inner core 360 of the straight stylet 314 may comprise nitinol or other metallic alloy or other flexible material. In some embodiments, the outer layer 365 may be comprised of a more rigid material, e.g., metallic or polymeric material (such as PEEK, polyurethane, PET, and/or the like).

FIG. 3WW illustrates an alternate embodiment of a distal portion of the straight stylet 314. The distal elongate shaft 370 may comprise an inner flexible, metal braided cable 380 (e.g., comprised of stainless steel, nitinol, or other metal alloy) extending from the proximal handle 316 and welded to the sharpened distal channeling tip 385 of the straight stylet 314, a metal hypotube 390 (e.g., comprised of stainless steel, nitinol, or other metal alloy) extending from the proximal handle 316 welded to the outside of the braided cable 380, and a flexible (e.g., polymeric, such as PEEK, polyurethane, PET, and/or the like) outer layer 395 extending from the hypotube 390 to the distal channeling tip 385. In some embodiments, the distal channeling tip 385 may have an end radius that has a more rounded profile. The straight stylet 314 may be flexible enough to bend to traverse the curved distal end portion 225 of the curved cannula 212 without significant friction. The straight stylet 314 may also be sufficiently rigid so as to maintain a straight path once the straight stylet 314 exits the open distal tip of the curved cannula 212.

FIGS. 3X-3Z illustrate an embodiment of an introducer drill 440 and its interaction with the introducer cannula 112. A kit or system of access instruments (e.g., a kit or kit module designed for accessing hard, or high-density, bone) may optionally include the introducer drill 440. FIG. 3X is a side view of an embodiment of the introducer drill 440. The introducer drill 440 may include a proximal handle 446 and an elongate drill shaft 447. The proximal handle 446 may comprise a generally T-shaped design and may comprise a soft-grip overmolding. The length of the elongate drill shaft 447 may be sized so as to extend from 20 mm to 35 mm beyond the open distal tip of the introducer cannula 112 when the introducer drill 440 is fully inserted within the introducer cannula 112. The elongate drill shaft 447 may include a solid proximal portion 448 and a fluted distal portion 449.

FIG. 3Y is a close-up perspective view of the fluted distal portion 449 of the introducer drill 440 of FIG. 3X. The fluted distal portion 449 may comprise a distal cutting tip 450 having an about 90 degree cutting angle. The drill flutes 452 of the fluted distal portion 449 may be adapted to taper away from the distal cutting tip 450 (which is a reverse taper or opposite the direction of taper of a typical drill bit) so as to facilitate improved bone chip packing within the open flute volume as bone chips and fragments are generated by operation of the introducer drill 440. The distal cutting tip 450 may have a point angle of between 65 and 75 degrees and a chisel edge angle of between 115 and 125 degrees. The flutes may advantageously be deeper and wider than typical drill bits because the elongate drill shaft 447 is supported by a rigid introducer cannula 112 surrounding at least a portion of the length of the elongate drill shaft (and a portion of the length of the fluted distal portion in most instances) during use. The drill flutes 452 may have a helix angle of between 12 degrees and 18 degrees (e.g., between 12 degrees and 14 degrees, between 13 degrees and 15 degrees, between 14 degrees and 16 degrees, between 15 degrees and 18 degrees, overlapping ranges thereof, or any value within the recited ranges). The fluted distal portion 449 may include two flutes having a length of between 70 mm and 85 mm.

The open flute volume of the fluted distal portion 449 may be advantageously configured to hold all or substantially all (e.g., more than 75%, more than 80%, more than 85%, more than 90%) of the significantly-sized bone chips or fragments removed by the introducer drill 440 as the introducer drill 440 is removed from the introducer cannula 112, thereby reducing the bone fragments left behind in the bone (e.g., vertebral body) or in the introducer cannula 112. In some embodiments, the open flute volume of the fluted distal portion 449 is adapted to hold about 2 ccs of bone. The fluted distal portion 449 may exhibit web tapering (e.g., increase in width or depth, or angle with respect to longitudinal axis of the flutes) along its length from distal to proximal (e.g., reverse taper). There may be no web taper for approximately the first 25 mm at the distal-most region. The web taper may then increase gradually until a maximum web taper is reached near the proximal end of the fluted distal portion 449 so as to facilitate pushing of the bone fragments or chip upward (or proximally) along the fluted distal portion 449. For example, the fluted distal portion 449 may have a negative draft (e.g., about 0.77 inch or 20 mm negative draft).

FIG. 3Z illustrates the introducer drill 440 fully inserted and engaged with the proximal handle 116 of the introducer cannula 112. The introducer drill 440 is sized so as to be inserted within the central opening 120 of the proximal handle 116 of the introducer cannula 112 and advanced through the central lumen 137 of the hypotube 118 of the introducer cannula 112. In some embodiments, the proximal handle 446 of the introducer drill 440 is configured to engage with the coupling or mating features 121 of the proximal handle 116.

### Retraction of the J-Stylet from the Introducer System

In low density bone, retraction of the curved stylet (e.g., J-stylet) 214 may be more difficult than in denser bone due to reduced cancellous bone support of the curved cannula 212. Less curved cannula support at the introducer cannula 112 exit can allow localized lateral deflection of the curved cannula 212 during removal of the curved stylet 214. This causes the introducer cannula exit to have greater interaction with the inner radius of the curved distal end portion 227 of the curved stylet 214 (through the curved cannula wall) as the stylet 214 is pulled into the introducer cannula 112 during removal. Transitions along the curved section inner radius can cause increased resistance during removal of the stylet 214. Therefore, increased flexibility of the curved distal end portion 227 and changes to the transition from the curved section to the distal channeling tip 228 can reduce this stylet / introducer system interaction to lower the stylet removal force.

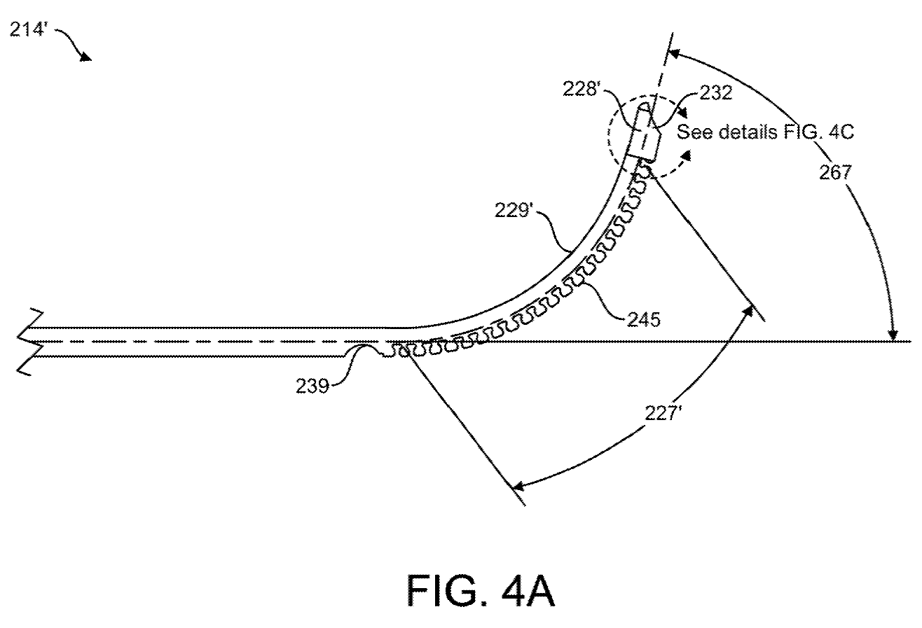

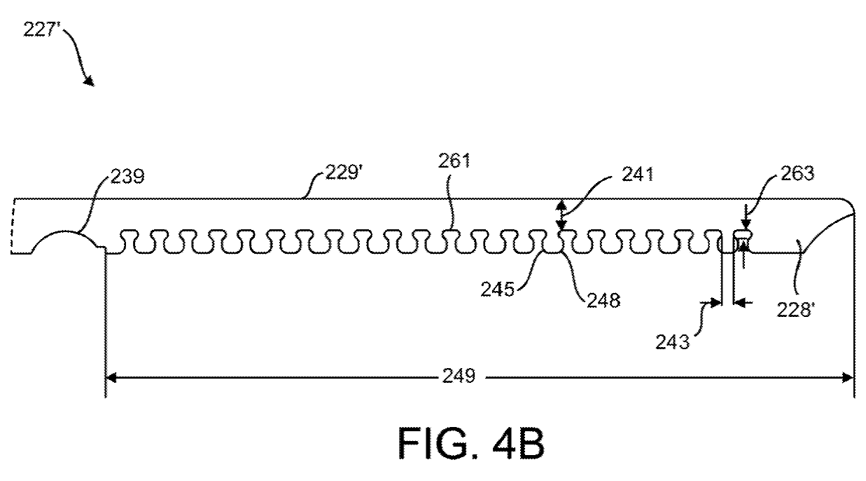

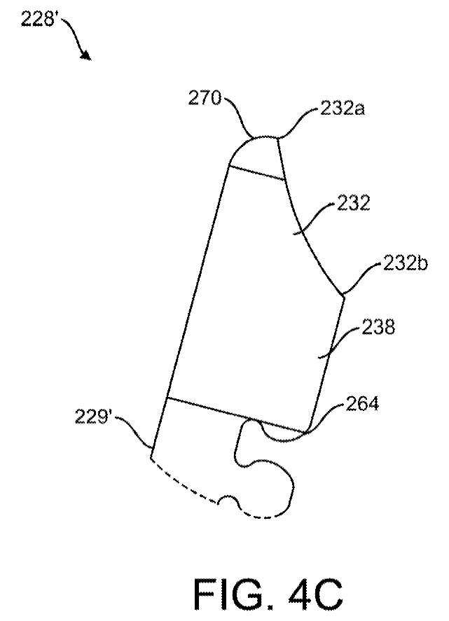

To address these challenges, FIGS. 4A-4C illustrate detailed views of an alternative embodiment of the J-stylet, denoted as stylet 214'. The stylet 214' incorporates all the basic features and functionality of stylet 214 described above, while including additional features and geometries detailed below. These additional features are particularly configured to enhance performance across varying bone densities and improve the overall reliability of the device during vertebral procedures. Unless specifically noted otherwise, all features and functions previously described for stylet 214 apply equally to stylet 214'. The following detailed description focuses on the distinguishing features and improvements incorporated into stylet 214'.

As shown in FIG. 4A, the disclosed stylet 214’ includes relief structures 245 along its outer curvature configured to maintain consistent cross-sectional profiles during deflection through varying bone densities. The structures 245 enables controlled deflection while supporting the walls of the curved cannula 212 during advancement and retraction procedures. This configuration addresses challenges associated with navigating through bone across different vertebral levels and densities.

FIG. 4A illustrates a side view configuration of stylet 214’ configured for controlled navigation through vertebral bone tissue. The stylet incorporates geometric features that enable consistent performance across varying bone densities while maintaining structural integrity throughout deflection procedures. The curved distal end portion 227’ is the deflectable region of the stylet. This portion transitions from a straight shaft configuration of the distal elongated shaft 218 into a predetermined curved geometry through manufacturing processes such as heat setting. The curved geometry enables controlled navigation through vertebral bodies while maintaining consistent deflection characteristics.

The curved distal end portion 227’ comprises a flexible curved section 229’ to provide the controlled deflection. This section incorporates material properties and geometric configurations that enable predictable deformation under applied loads while preserving structural integrity. The flexible curved section 229’ maintains its mechanical properties across varying bone densities through specific material selection and processing parameters.

The flexible curved section 229’ comprises a plurality of relief structures 245 that extend along the outer curvature in a uniform pattern. These structures 245 comprise a plurality of projections and recesses creating plurality of radially-oriented ridges that are perpendicular in the length direction of the curved distal end portion 227’ and work to maintain the cross-sectional profile of the stylet during deflection. The relief structures 245 may look like “mushroom” shapes and create localized areas of increased flexibility, enabling the curved section 229’ to bend in a controlled manner. At the same time, the projections (ridges) of the relief structure provide support to the curved cannula wall, preventing unwanted deformation or kinking. Each of the plurality of relief structures 245 incorporates filleted edge profiles that establish smooth transitions between the projecting and recessed surfaces. These fillets may range from 0.005 to 0.015 inches in radius, reducing particulate generation while enabling smooth engagement with the curved cannula wall during advancement and retraction procedures through manufacturing processes including such as electropolishing. The smooth transitions prevent the edges from digging into or abrading the cannula wall. In some embodiments, the edges may alternatively utilize chamfered or rounded profiles to achieve similar smooth transitions.

The quantity, spacing, and geometry of the relief structures 245 may be selected to optimize the flexibility, stiffness, and support of the curved distal end portion 227’. As illustrated in FIG. 4A, the curved section 229’ may comprises twenty-two relief structures uniformly spaced along the outer curvature. This configuration creates discrete flex points that enable controlled, balanced bending. However, the quantity of relief structures may be decided based on the desired flexibility while maintaining structural integrity. The spacing between adjacent relief structures 245 may range from 0.010 to 0.020 inches to achieve uniform curvature during deflection.

A distal channeling tip 228’ extends from the curved section 229’ and forms the leading end of the stylet for penetrating bone. The distal channeling tip 228’ incorporates a hollow grind tip with a multi-angled geometry at its distal-most point. This configuration may enable better puncture performance and generate a more predictable curved path through the bone tissue. The distal channeling tip 228’ may also feature a wire EDM-cut (electrical discharge machining) configuration with a reduced profile at its distal-most tip. This smaller tip may enable the tip to more readily begin channeling and engage with the cancellous bone, enhancing puncture performance. The distal channeling tip 228’ can be seen and discussed in more detail in FIG. 4C.

The curved distal end portion 227’ may also have an overall bend angle 267 that defines the curved trajectory of the stylet during navigation through bone. The bend angle 267 may be approximately 75 degrees to facilitate access to the posterior regions of vertebral bodies across multiple levels and densities of bone. The bend angle may range anywhere from 65 to 80 degrees to accommodate various anatomical approaches and access requirements.

Furthermore, an alignment feature 239 is disposed at the proximal end of the curved distal end portion 227’. This feature 239 has a semi-circular profile that interfaces with injection molding tooling or other tools during manufacturing of the stylet. The alignment feature 239 enables consistent orientation of the curved section 229’ and distal channeling tip 228’ relative to the handle, reproducing the geometric relationships within the curved distal end portion 227’ enhances the reliability and repeatability of the stylet's navigational performance.

The curved distal end portion 227’ may also have a specified thickness 241 to achieve the desired flexibility and resilience during use. The thickness balances the need for lateral flexibility with sufficient columnar strength to resist buckling or permanent deformation.

The material selection and processing of the curved distal end portion 227’ may also influence its mechanical properties and performance. In an example embodiment, the curved section 229’ may be formed from nickel titanium alloy (nitinol) with specified heat treatment parameters. These heat treatments may optimize the shape memory and elastic characteristics of the nitinol, enabling the stylet to navigate through high density bone without experiencing plastic deformation. Other flexible, resilient, biocompatible materials may be used in alternative embodiments.

Although stylet 214’ utilizes uniform spacing between the relief structures 245, alternative configurations may incorporate variable spacing to fine-tune the flexibility along the length of the curved distal end portion 227’. For example, the spacing may decrease from a proximal end to a distal end of the curved section 229’. This configuration would provide greater flexibility closer to the interface between the curved section 229’ and distal channeling tip 228’. By enhancing distal flexibility, the curved portion 227’ may more readily conform to the central lumen of the curved cannula during retraction procedures, thereby reducing contact forces and minimizing damage to the cannula wall.

In some embodiments, the relief structures 245 may have an asymmetric geometry to provide preferential bending characteristics. For example, the projections on the outer surface of the curved section 229’ may have a different shape or height. This asymmetry would bias the curved portion 227’ to bend in a desired direction and resist deflection in non-preferred directions. Asymmetric relief structures 245 may be beneficial in certain anatomical applications where the stylet navigates through non-uniform bone or around sensitive tissues.

The disclosed stylet 214’ incorporating relief structures 245 overcomes challenges associated with navigating through bone of varying densities across multiple vertebral levels. The flexible curved section 229’ provides controlled, predictable deflection while maintaining structural integrity. The relief structures 245 with filleted edges maintain consistent contact with the curved cannula wall, preventing damage and enabling smooth advancement and retraction. The distal channeling tip 228’ optimizes bone penetration and facilitates the desired curved trajectory through the vertebral bodies. These features combine to enhance the overall performance and reliability of the stylet, improving patient outcomes in vertebral augmentation and other spinal intervention procedures.

FIG. 4B provides a detailed side view of the curved distal end portion 227’ of the stylet 214’ as shown in a conformal straight position (e.g. positioned within the curved and introducer cannula), illustrating the geometric features that enable its controlled deflection and navigation through varying bone densities. The curved distal end portion 227’ includes a flexible curved section 229’ with a plurality of relief structures 245 extending along its outer curvature.

The relief structures 245 comprise a plurality of projections or ridges separated by a plurality of recessed regions or troughs 261. These structures are uniformly spaced along the length of section 249 to provide consistent flexibility and support. As shown in FIG. 4B, section 249 includes twenty-two (22) relief structures 245. However, the number of relief structures may vary depending on the desired characteristics of the curved distal end portion 227’. In some embodiments, the number of relief structures may range from eighteen (18) to twenty-six (26).

Each relief structure 245 has a geometry configured to optimize the performance of the stylet 214’ during advancement and retraction through bone. The ridges of the relief structures 245 have a generally a “horseshoe” cross-section with filleted edges 248 that blend smoothly into the adjacent recessed regions 261. The filleted edges 248 help to reduce stress concentrations and minimize the generation of particulate during contact. The fillets 248 may have a radius ranging from 0.005 inches to 0.015 inches. In some embodiments, the fillet radius may measure 0.012 inches. The width of each relief structure ridge 243, is selected to provide sufficient structural integrity while enabling the desired flexibility characteristics. The width 243 may range from 0.015 inches to 0.025 inches. In some embodiments, the width 243 may measures 0.020 inches with a tolerance of ±0.002 inches. The dimension may be selected to offer an optimal balance between lateral flexibility and columnar strength, enabling the stylet 214’ to navigate effectively through high density bone without pinching or damaging the inner lumen of the curved cannula 212.

The recessed regions or troughs 261 between the relief structure ridges also enable the flexibility and navigational capabilities of the curved distal end portion 227’. Depth of the troughs 263 can also directly influence the amount of material removed from the curved section 229’ and, consequently, its bending stiffness. The depth 263 of the troughs may range from 0.010 inches to 0.020 inches. In some embodiments, the trough depth may measure 0.014 inches with a tolerance of ±0.002 inches. This depth provides a balance between flexibility and structural integrity, enabling the stylet 214 to conform to the curvature of the cannula 212 while resisting excessive deformation or buckling.

The shape of the recessed regions or troughs 261 are also configured to facilitate smooth bending and minimize stress concentrations. As shown in FIG. 4B, the troughs have a contour that resembles interlocking puzzle pieces. This geometry features a series of alternating curved and straight segments that blend with the adjacent relief structure ridges. The curved segments create a continuous and smooth transition between the two features. The straight segments, oriented perpendicular to the longitudinal axis of the stylet 214’, provide a uniform depth and width to the troughs, enabling consistent flexibility along the length of the curved section 229’. The puzzle piece-like geometry of the recessed regions 261 demonstrates the nature of the curved and straight segments that help to distribute stresses more evenly across the relief structures 245, reducing the risk of localized strain or fatigue. Additionally, the smooth transitions between the ridges and troughs minimize the potential for stress risers or crack initiation points, enhancing the overall durability and reliability of the curved distal end portion 227’.

The overall stiffness or rigidity of the curved distal end portion 227’ may also influenced by several factors, including the number and spacing of the relief structures 245 as well as the thickness 241 of the curved section 229’. The thickness 241 may range from 0.050 inches to 0.060 inches. In some embodiments, the thickness may measure 0.054 inches with a tolerance of ±0.003 inches. This thickness provides the necessary flexibility for the stylet 214’ to conform to the curved path within the central lumen of the curved cannula 212 while resisting buckling or excessive deformation under the forces required to penetrate bone. The stiffness of the curved section 229’ may also be tailored to suit different bone densities or specific procedural requirements by adjusting the thickness 241. In some embodiments, a slightly thinner thickness, such as 0.051 inches, may be desirable for procedures involving softer or more porous bone. The reduced stiffness enables the stylet 214 to deflect more easily and follow the curved trajectory with less resistance. Conversely, a slightly thicker thickness, such as 0.057 inches, may be preferred for denser bone. The increased rigidity helps to maintain the curved path and resist deviations caused by the higher penetration forces.

The length of section 249 is another parameter that may influence the navigational capabilities of the stylet 214. In some embodiments, this length may be in a range of 1.250 inches to 1.400 inches. In some embodiments, the length may be of 1.285 inches with a tolerance of +0.010 inches, -0.015 inches. This length may provide sufficient flexibility to traverse the curved cannula 212 while maintaining enough columnar strength to resist excessive deflection or buckling during advancement. Variations in the length of section 249 can affect the steerability and trajectory of the stylet 214’ within the vertebral body. A slightly shorter length, such as 1.270 inches, may be advantageous in scenarios where a tighter turning radius is required to navigate around obstacles or access specific regions. The reduced length concentrates the bending closer to the distal tip 228’, enabling for more precise control. On the other hand, a slightly longer length, such as 1.300 inches, may be beneficial when a more gradual curve is desired. The increased length distributes the bending moment over a larger area, potentially reducing stresses on the stylet 214’ and curved cannula 212.

The length of section 249 may also serve as mechanical safety feature to prevent overdrive during the procedure. Overdrive occurs when physicians continue to mallet once they've reached full depth with their tool, which can cause the introducer cannula to advance straight ahead and create a kink in the curved cannula. These kinks can potentially lead to PEEK material being left behind in the patient. To address this overdrive risk, the length of section 249 may be configured to extend approximately 5 millimeters beyond the distal end of the introducer cannula when the device is fully inserted. The length of section 249 serves to minimize the likelihood of overdrive during use. As the section reaches its intended insertion depth, its structural resistance to bending creates a mechanical impedance that resists further advancement. This resistance causes the J-stylet to be partially ejected from the introducer cannula when excessive force is applied, thereby providing tactile feedback to the operator and discouraging continued malleting. This mechanism reduces the risk of the introducer cannula advancing in a linear path that could potentially create a kink in the curved cannula. This length of section 249 essentially functions as a shock absorber. If overdrive occurs, the straight section helps absorb the impact forces and resist kinking of the curved cannula.

The length of section 249 may also help to minimize interference and binding between the stylet 214’ and the introducer cannula 112, particularly during initial insertion or final retraction steps. The constant cross-section of the length of section 249 enables it to slide smoothly within the distal portion of the introducer cannula 112 without experiencing abrupt changes in flexibility or stiffness. This smooth transition reduces the risk of the stylet 214’ catching or snagging on the internal surface of the curved cannula, which could potentially cause damage or disruption to the surrounding tissue.

The material selection and processing techniques used in the manufacture of the curved section 229’ may also influence its mechanical properties and performance. The curved section 229’ may be made from a nickel-titanium alloy, commonly known as nitinol. This shape memory alloy exhibits elastic behavior, enabling it to undergo large deformations without permanent damage or plastic deformation. The elastic properties of nitinol enable the curved distal end portion 227’ to navigate through high density bone while maintaining its pre-defined curvature and shape.

To achieve the desired mechanical characteristics, the nitinol curved section 229’may undergo a series of precise heat treatment and forming processes. After heat treatment, the curved section 229’ may undergo additional surface processing steps to improve its smoothness and biocompatibility. One common technique is electropolishing. Electropolishing helps to reduce surface irregularities, burrs, and sharp edges that could potentially cause damage or irritation to the surrounding tissue. The smooth, polished surface of the curved section 229’ also facilitates its passage through the curved cannula 212 by minimizing friction and interference.

In some embodiments, the curved section 229’ may incorporate additional features or modifications to enhance its performance or adaptability. For example, the relief structures 245 may be arranged in a non-uniform pattern, with variable spacing or geometry along the length of the curved section 229’. This variable arrangement could be used to fine-tune the flexibility profile of the curved distal end portion 227’, providing increased stiffness in certain regions and greater flexibility in others. Such a configuration may be advantageous in scenarios where the stylet 214’ needs to navigate through bone of varying density or encounter specific anatomical obstacles.

In some embodiments, the curved section 229’ may incorporate use of composite materials or reinforcing elements in the construction of the curved section 229’. For example, the nitinol structure could be supplemented with a thin layer of a polymer or elastomer to provide additional dampening or vibration absorption. Alternatively, the curved section 229’ could incorporate discrete reinforcing fibers or wires to enhance its tensile strength or resistance to kinking.

The curved section 229’ of the stylet 214’ incorporates a range of geometric features and material properties that enable its controlled navigation through bone of varying density. The relief structures 245 with filleted edges 248 and puzzle piece-like recessed regions 261 provide flexibility and support, while the thickness 241 and length of the curved section 229’ influence the overall stiffness and trajectory of the stylet.

Referring now to FIG. 4C, a detailed view of the distal portion of the stylet 214’, specifically focusing on the geometry and features of the distal channeling tip 228’ is shown. The distal channeling tip 228’ is located at the distal end of the flexible curved section 229’ and is configured to facilitate penetration and navigation through bone tissue.

The distal channeling tip 228’ includes a curved surface 232 that lies between points 232a and 232b creating a specific cutting profile for the distal channeling tip 228’. Rather than having a straight angled surface between these points, the curved profile enables a variable cutting angle during bone penetration. As the tip first contacts bone, the leading edge at 232a initiates the penetration, while the curved surface 232 then engages progressively with the bone material. This progressive engagement helps control the rate and direction of bone cutting compared to a straight cutting surface. The curvature of surface 232 also helps guide the direction of cut bone material flow during penetration. Rather than bone material potentially building up against a flat surface, the curved profile helps direct displaced bone material away from the cutting edge. Below the curved surface 232, the distal channeling tip 228’ features a straight section 238. This straight section 238 extends proximally from the point 232b and may contribute to the overall stability and trajectory control of the distal channeling tip 228’ as it advances through bone tissue.

At the distal end of the straight section 238, the distal channeling tip 228’ incorporates a rounded or filleted transition 264. This filleted transition 264 provides a smooth connection between the straight section 238 and the distal-most portion of the channeling tip 228’. The presence of this rounded transition may help to distribute stresses and reduce the risk of damage to the tip during use. The distal-most portion of the channeling tip 228’ also features a rounded tip 270. In some embodiments, the rounded tip may have a radius of approximately 0.025 inches. This rounded tip 270 helps to protect from getting caught as the stylet is advanced through the bone tissue. The rounded geometry enables for a smooth passage, minimizing the risk of tissue damage or particulate generation. The rounded tip 270 may also ensure the device can advance through the curved PEEK cannula without scraping, scoring, and damaging the material. The rounded tip may feature a wire EDM cut to create a slightly sharper angle at the tip, enhancing its ability to catch and penetrate bone more readily.

The specific dimensions, angles, and radii of the various components of the distal channeling tip 228’, such as the curved surface 232, straight section 238, filleted transition 264, and rounded tip 270, may be optimized based on factors such as the intended use, desired performance characteristics, and the material properties of the stylet. Variations in the geometry of the distal channeling tip 228’ may be employed to suit different clinical requirements or to enhance specific aspects of its performance. For example, the curvature of the curved surface 232 may be adjusted to provide a more gradual or abrupt transition. Similarly, the length of the straight section 238 or the radius of the filleted transition 264 may be modified to achieve desired stability, strength, or stress distribution characteristics.

In some embodiments, the distal channeling tip 228’ may feature different shapes or configurations to address specific clinical needs. For example, the tip may incorporate a beveled or angled configuration to facilitate steering or directional control during advancement. Additionally, the tip may feature multiple facets or cutting edges to enhance bone penetration efficiency.

The distal channeling tip 228’, with its combination of geometric features, including the curved surface 232, straight section 238, filleted transition 264, and rounded tip 270, is configured to efficiently penetrate and navigate through bone tissue while minimizing the risk of damage or complications. By balancing these elements, the stylet 214’ can access targeted sites while maintaining a safe profile.

The features and improvements detailed in FIGS. 4A-4C address specific challenges encountered when using the J-stylet in different bone densities. The relief structures 245 help prevent the curved cannula from deforming under lateral forces, while maintaining support along the curved path. The distal channeling tip 228’, with its curved surface 232 and radius-tipped profile, provides effective bone penetration capabilities while enabling controlled removal. The bend angle and dimensional specifications support procedures and placement while managing removal forces. These elements work together to enable controlled navigation through bone tissue while reducing the forces required during stylet removal. This configuration of the J-stylet achieves its intended bone access function with improved removal characteristics.

### Modulation of Tissue in Bone

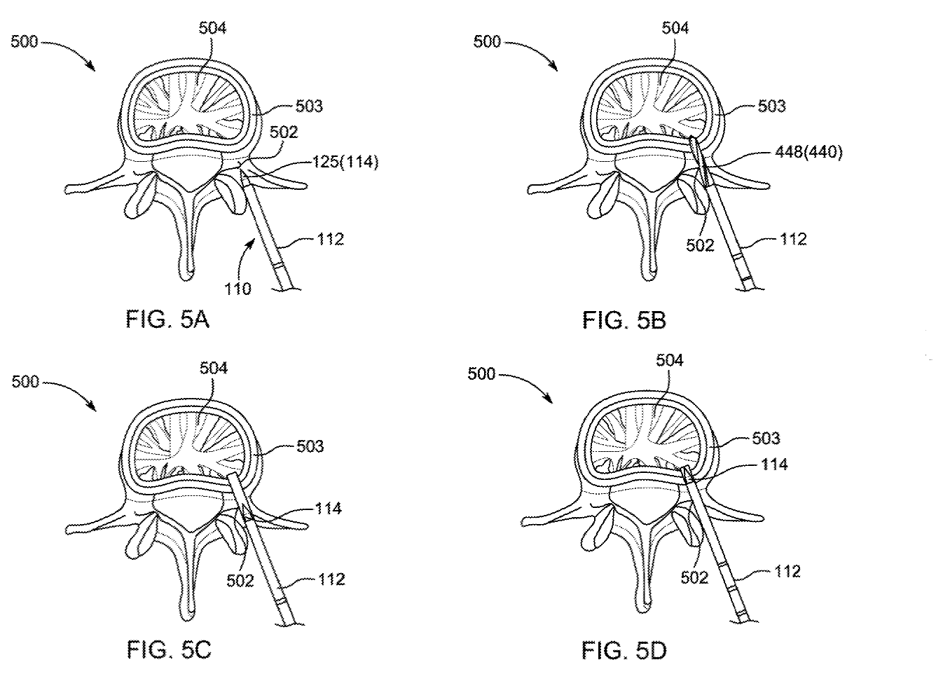

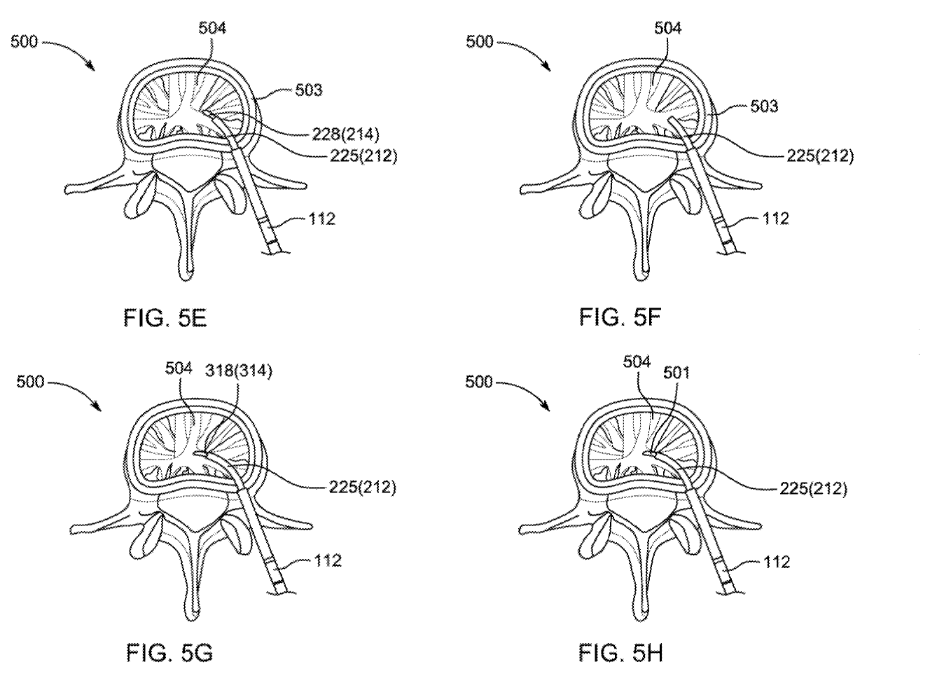

FIGS. 5A – 5H illustrate an embodiment of steps of a method of using the access tools to facilitate access to a location within a vertebral body 500 for treatment (e.g., modulation of intraosseous nerves, such as a basivertebral nerve, bone cement delivery for treatment of vertebral fractures, and/or ablation of bone tumors). With reference to FIG. 5A, the distal portion of the introducer assembly 110 (including the distal tip 125 of the introducer stylet 114 and the distal tip of the introducer cannula 112) are inserted through a pedicle 502 adjacent the vertebral body 500 by malleting on the proximal handle of the introducer stylet 114 after insertion and aligned engagement of the introducer stylet 114 within the introducer cannula 112.

In accordance with several embodiments, the method may optionally include removing the introducer stylet after initial penetration into the pedicle 502 (for example, if the operator can tell that the density of the bone is going to be sufficiently dense or hard that additional steps and/or tools will be needed to obtain a desired curved trajectory to access a posterior portion (e.g., posterior half) of the vertebral body 500. With reference to FIG. 5B, the method may optionally include inserting the introducer drill 440 into and through the introducer cannula 112 to complete the traversal of the pedicle 502 and penetration through a cortical bone 503 region of the vertebral body 500 until a cancellous bone region 504 of the vertebral body 500 is reached. The introducer drill 440 may be advanced into the cancellous bone region 504 (especially if the cancellous bone region 504 is determined to be sufficiently hard or dense) or the advancement may stop at the border between the cortical bone region 503 and the cancellous bone region 504. In some embodiments, this step may involve both rotating the introducer drill 440 and malleting on the proximal handle 446 of the introducer drill 440 or simply rotating the introducer drill 440 without malleting on the proximal handle 446. With reference to FIG. 5C, the introducer drill 440 may be removed and the introducer stylet 114 may be re-inserted within the introducer cannula 112. In some embodiments, the introducer may have a pointed end to match the pointed end of the drill tip. With reference to FIG. 5D, the introducer assembly 110 may then be malleted so as to advance the distal tip 122 of the introducer cannula 112 to the entry site into (or within) the cancellous bone region 504 of the vertebral body 500. The introducer stylet 114 may then be removed from the introducer cannula 112. In some embodiments, the scallop section 133 of the introducer stylet 114 are aligned to the beginning of the cancellous bone region, as such distal tip 122 is may not normally in bone region 504.

The curved cannula assembly 210 may then be inserted within the introducer cannula 112 with the gear wheel 221 in the distal-most position so as to prevent inadvertent advancement of the curved cannula assembly 210 out of the open distal tip 122 of the introducer cannula 112 prematurely. With reference to FIG. 5E, after rotation of the gear wheel 221 to a more proximal position, the curved cannula assembly 210 can be malleted so as to advance the collective curved distal end portions of the curved cannula assembly 210 together out of the distal tip 122 of the introducer cannula 112 and along a curved path within the cancellous bone region 504. With reference to FIG. 5F, the J-stylet 214 may then be removed from the curved cannula 212, with the curved cannula 212 remaining in position. In accordance with several embodiments, the path formed by the prior instruments may advantageously allow the curved cannula assembly 210 to have a head start and begin curving immediately upon exiting the open distal tip 122 of the introducer cannula 112. In some embodiments, the J-stylet may be that of stylet 214’ and incorporate relief structures 245 along the outer curvature of its curved distal end portion 227’. These relief structures 245 help in the navigation and positioning of the curved cannula 212 within the cancellous bone region 504. As the J-stylet 214' is advanced through the curved cannula 212, the relief structures 245 provide controlled flexibility, bending, and support, enabling the stylet to maintain its predetermined curved trajectory while minimizing the risk of buckling or unintended deformation. The geometry and spacing of the relief structures 245 are configured to distribute forces along the length of the curved distal end portion 227, enabling a smooth and predictable advancement through the bone tissue. The bending may have a bend angle that ranges anywhere from 65 to 80 degrees to accommodate various anatomical approaches and access requirements Furthermore, the relief structures 245 of the J-stylet 214' facilitate its smooth retraction from the curved cannula 212 once the desired positioning is achieved. The presence of these structures reduces the contact area between the outer surface of the stylet and the inner wall of the cannula, minimizing friction and potential binding during removal. This ease of retraction is beneficial in minimally invasive procedures, as it enables the efficient exchange of instruments through the curved cannula 212 without disturbing its placement or causing undue stress on the surrounding tissue.

With reference to FIG. 5G, if a further straight path beyond the curved path is desired to reach a target treatment location, the straight stylet 314 may be inserted through the curved cannula 212 such that the distal channeling tip 319 of the straight stylet extends beyond the open distal tip of the curved cannula 212 and along a straight path toward the target treatment location (e.g., a basivertebral nerve trunk or basivertebral foramen). In some embodiments, the straight stylet 314 may not be needed and this step may be skipped.

With reference to FIG. 5H, a treatment device 501 (e.g., a flexible bipolar radiofrequency probe) may be inserted through the curved cannula 212 (after removal of the straight stylet 314 if used) and advanced out of the open distal tip of the curved cannula 212 to the target treatment location. The treatment device 501 may then perform the desired treatment. For example, if the treatment device 501 is a radiofrequency probe, the treatment device 501 may be activated to ablate intraosseous nerves (e.g., a basivertebral nerve) or a tumor within the vertebral body 500. Bone cement or other agent, or a diagnostic device (such as a nerve stimulation device or an imaging device to confirm ablation of a nerve) may optionally be delivered through the curved cannula 212 after the treatment device 501 is removed from the curved cannula 212.

At certain levels of the spine (e.g., sacral and lumbar levels) and for certain patient spinal anatomies that require a steeper curve to access a desired target treatment location within the vertebral body, a combination curette/curved introducer may first be inserted to start a curved trajectory (e.g., create an initial curve or shelf) into the vertebra. The curette may have a pre-curved distal end portion or be configured such that the distal end portion can be controllably articulated or curved (e.g., manually by a pull wire or rotation of a handle member coupled to one or more pull wires coupled to the distal end portion or automatically by a robotic or artificial intelligence driven navigation system). The combination curette/curved introducer may then be removed and the outer straight cannula and inner curved cannula/curved stylet assembly may then be inserted to continue the curve toward the target treatment location.

In accordance with several implementations, any of the access tools (e.g., cannula or stylet) or treatment devices may comprise a rheological and/or magnetizable material (e.g., magnetorheological fluid) along a distal end portion of the access tool that is configured to be curved in situ after insertion to a desired location within bone (e.g., vertebra). A magnetic field may be applied to the distal end portion of the access tool and/or treatment device with the magnetizable fluid or other material and adjusted or varied using one or more permanent magnets or electromagnets to cause the distal end portion of the access tool and/or treatment device to curve toward the magnetic field. In some implementations, a treatment probe may include a magnetic wire along a portion of its length (e.g., a distal end portion). Voltage applied to the magnetic wire may be increased or decreased to increase or decrease a curve of the magnetic wire. These implementations may advantageously facilitate controlled steering without manual pull wires or other mechanical mechanisms. The voltage may be applied by instruments controlled and manipulated by an automated robotic control system.

The treatment devices (e.g., treatment probes) may be any device capable of modulating tissue (e.g., nerves, tumors, bone tissue). Any energy delivery device capable of delivering energy can be used (e.g., RF energy delivery devices, microwave energy delivery devices, laser devices, infrared energy devices, other electromagnetic energy delivery devices, ultrasound energy delivery devices, and the like). The treatment device 501 may be an RF energy delivery device. The RF energy delivery device may include a bipolar pair of electrodes at a distal end portion of the device. The bipolar pair of electrodes may include an active tip electrode and a return ring electrode spaced apart from the active tip electrode. The RF energy delivery device may include one or more temperature sensors (e.g., thermocouples, thermistors) positioned on an external surface of, or embedded within, a shaft of the energy delivery device. The RF energy delivery device may not employ internally circulating cooling, in accordance with several implementations.

In some implementations, water jet cutting devices may be used to modulate (e.g., denervate) nerves. For example, a water jet cutter may be configured to generate a very fine cutting stream formed by a very high-pressure jet of water. For example, the pressure may be in the range of 15 MPa to 500 MPa (e.g., 15 MPa to 50MPa, 30 MPa – 60 MPa, 50 MPa – 100 MPa, 60 MPa – 120 MPa, 100 MPa – 200 MPa, 150 MPa – 300 MPa, 300 MPa – 500 MPa, overlapping ranges thereof, or any value within the recited ranges). In some implementations, a chemical neuromodulation tool injected into a vertebral body or at an endplate may be used to ablate or otherwise modulate nerves or other tissue. For example, the chemical neuromodulation tool may be configured to selectively bind to a nerve or endplate. In some implementations, a local anesthetic (e.g., liposomal local anesthetic) may be used inside or outside a vertebral body or other bone to denervate or block nerves. In some implementations, brachytherapy may be used to place radioactive material or implants within the vertebral body to deliver radiation therapy sufficient to ablate or otherwise denervate the vertebral body. In some implementations, chymopapain injections and/or condoliase injections may be used (e.g., under local anesthesia). Phototherapy may be used to ablate or otherwise modulate nerves after a chemical or targeting agent is bound to specific nerves or to a vertebral endplate.

In accordance with several implementations, thermal energy may be applied within a cancellous bone portion (e.g., by one or more radiofrequency (RF) energy delivery instruments coupled to one or more RF generators) of a vertebral body. The thermal energy may be conducted by heat transfer to the surrounding cancellous bone, thereby heating up the cancellous bone portion. In accordance with several implementations, the thermal energy is applied within a specific frequency range and having a sufficient temperature and over a sufficient duration of time to heat the cancellous bone such that the basivertebral nerve extending through the cancellous bone of the vertebral body is modulated. In several implementations, modulation comprises permanent ablation or denervation or cellular poration (e.g., electroporation). In some implementations, modulation comprises temporary denervation or inhibition. In some implementations, modulation comprises stimulation or denervation without necrosis of tissue.

For thermal energy, temperatures of the thermal energy may range from about 70 to about 115 degrees Celsius (e.g., from about 70 to about 90 degrees Celsius, from about 75 to about 90 degrees Celsius, from about 83 to about 87 degrees Celsius, from about 80 to about 100 degrees Celsius, from about 85 to about 95 degrees Celsius, from about 90 to about 110 degrees Celsius, from about 95 to about 115 degrees Celsius, or overlapping ranges thereof). The temperature ramp may range from 0.1 – 5 degrees Celsius/second (e.g., 0.1 – 1.0 degrees Celsius/second, 0.25 to 2.5 degrees Celsius/second, 0.5 – 2.0 degrees Celsius/second, 1.0 – 3.0 degrees Celsius/second, 1.5 – 4.0 degree Celsius/second, 2.0 – 5.0 degrees Celsius/second). The time of treatment may range from about 10 seconds to about 1 hour (e.g., from 10 seconds to 1 minute, 1 minute to 5 minutes, from 5 minutes to 10 minutes, from 5 minutes to 20 minutes, from 8 minutes to 15 minutes, from 10 minutes to 20 minutes, from 15 minutes to 30 minutes, from 20 minutes to 40 minutes, from 30 minutes to 1 hour, from 45 minutes to 1 hour, or overlapping ranges thereof). Pulsed energy may be delivered as an alternative to or in sequence with continuous energy. For radiofrequency energy, the energy applied may range from 350 kHz to 650 kHz (e.g., from 400 kHz to 600 kHz, from 350 kHz to 500 kHz, from 450 kHz to 550 kHz, from 500 kHz to 650 kHz, overlapping ranges thereof, or any value within the recited ranges, such as 450 kHz ± 5 kHz, 475 kHz ± 5 kHz, 487 kHz ± 5 kHz). A power of the radiofrequency energy may range from 5 W to 30 W (e.g., from 5 W to 15 W, from 5 W to 20 W, from 8 W to 12 W, from 10 W to 25 W, from 15 W to 25 W, from 20 W to 30 W, from 8 W to 24 W, and overlapping ranges thereof, or any value within the recited ranges). In accordance with several implementations, a thermal treatment dose (e.g., using a cumulative equivalent minutes (CEM) 43 degrees Celsius thermal dose calculation metric model) is between 200 and 300 CEM (e.g., between 200 and 240 CEM, between 230 CEM and 260 CEM, between 240 CEM and 280 CEM, between 235 CEM and 245 CEM, between 260 CEM and 300 CEM) or greater than a predetermined threshold (e.g., greater than 240 CEM). The CEM number may represent an average thermal cumulative dose value at a target treatment region or location and may represent a number that expresses a desired dose for a specific biological end point. Thermal damage may occur through necrosis or apoptosis.

Cooling may optionally be provided to prevent surrounding tissues from being heated during the nerve modulation procedure. The cooling fluid may be internally circulated through the delivery device from and to a fluid reservoir in a closed circuit manner (e.g., using an inflow lumen and an outflow lumen). The cooling fluid may comprise pure water or a saline solution having a temperature sufficient to cool electrodes (e.g., 2 – 10 degrees Celsius, 5 – 10 degrees Celsius, 5 – 15 degrees Celsius). Cooling may be provided by the same instrument used to deliver thermal energy (e.g., heat) or a separate instrument. In accordance with several implementations, cooling is not used.

In some implementations, ablative cooling may be applied to the nerves or bone tissue instead of heat (e.g., for cryoneurolysis or cryoablation applications). The temperature and duration of the cooling may be sufficient to modulate intraosseous nerves (e.g., ablation, or localized freezing, due to excessive cooling). The cold temperatures may destroy the myelin coating or sheath surrounding the nerves. The cold temperatures may also advantageously reduce the sensation of pain. The cooling may be delivered using a hollow needle under fluoroscopy or other imaging modality.

In some implementations, one or more fluids or agents may be delivered to a target treatment site to modulate a nerve. The agents may comprise bone morphogenetic proteins, for example. In some implementations, the fluids or agents may comprise chemicals for modulating nerves (e.g., chemoablative agents, alcohols, phenols, nerve-inhibiting agents, or nerve stimulating agents). The fluids or agents may be delivered using a hollow needle or injection device under fluoroscopy or other imaging modality.

One or more treatment devices (e.g., probes) may be used simultaneously or sequentially. For example, the distal end portions of two treatment devices may be inserted to different locations within a vertebral body or other bone or within different vertebral bodies or bones. Radiofrequency treatment probes may include multiple electrodes configured to act as monopolar, or unipolar, electrodes or as pairs of bipolar electrodes. The treatment device(s) may also be pre-curved or curveable such that the curved stylet is not needed or may have sharp distal tips such that additional sharpened stylets are not needed. In some implementations, any or all of the access tools and the treatment devices are MR-compatible so as to be visualized under MR imaging.

The one or more treatment devices (e.g., probes such as radiofrequency probes, treatment device 501 of a kit or system) may include an indicator configured to alert a clinician as to a current operation state of the treatment device. For example, the indicator may include a light ring disposed along a length of, and extending around a circumference of, the treatment device. The light ring may be configured to light up with different colors and/or exhibit other visible effects (e.g., pulsing on and off with certain patterns). The one or more treatment devices may also be configured to provide audible alerts (e.g., beeps having a certain frequency or intonation) corresponding to different operational states. In one implementation, the light ring may be dark or not lit up when the treatment device is not connected to a radiofrequency generator or not ready for RF energy delivery. The light ring may pulse at a first rate (e.g., 1 pulse every 2-3 seconds) to indicate an operational state in which the treatment device and generator system are ready to initiate RF energy delivery. The light ring may be continuously lit up to indicate an operational state in which the treatment device is actively delivering RF energy. The light ring may pulse at a second rate different than (e.g., faster than, slower than) the first rate to indicate an operational state in which an error has been detected by the generator or if a particular treatment parameter is determined to be outside an acceptable range of values. In one implementation, the second rate is greater than the first rate (e.g., 2 pulses per second). Haptic feedback may also be provided to the clinician for at least some of the operational states to provide a further alert in addition to a visible alert.

In some implementations, the treatment device (e.g., treatment device 501) includes a microchip that is pre-programmed with treatment parameters (e.g., duration of treatment, target temperature, temperature ramp rate). Upon electrical connection of the treatment device to the generator, the treatment parameters are transmitted to the generator and displayed on a display of the generator to provide confirmation of desired treatment to a clinician.

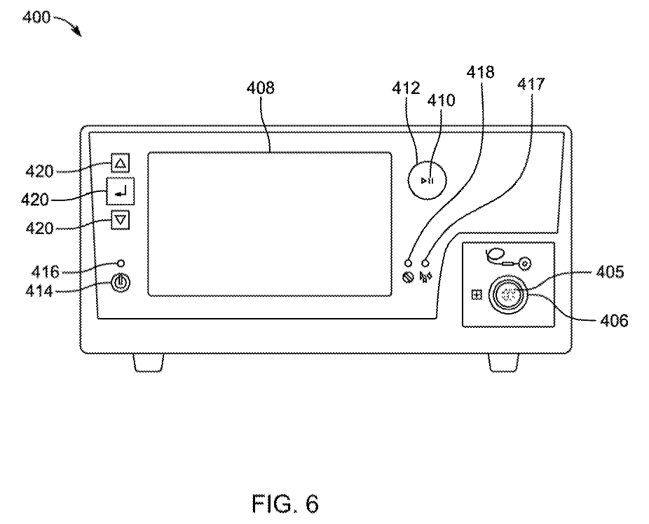

FIG. 6 illustrates a front view of an embodiment of a generator 400 (e.g., radiofrequency energy generator). The generator 400 includes an instrument connection port 405 to which a treatment device (e.g., RF energy delivery probe) may be connected. The generator 400 may be configured for use without a neutral electrode (e.g., grounding pad). The instrument connection port 605 is surrounded by an indicator light 406 configured to illuminate when the treatment device is properly connected to the instrument connection port 405. As shown, the indicator light 406 may comprise a circular LED indicator light. The indicator light 406 may be configured to continuously illuminate in a solid color (e.g., white, blue, green) when a treatment device is connected to the instrument connection port 405. The indicator light 406 may flash at a first pulsing rate (e.g., 1 Hz) to prompt a clinician to connect the treatment device to the instrument connection port 405. The indicator light 406 may flash at a second pulsing rate different than (e.g., faster than) the first pulsing rate (e.g., 2 Hz, 3 Hz, 4 Hz) to indicate an error condition.

The generator 400 also includes a display 408 configured to display information to the clinician or operator. During startup and use, the current status of the generator 400 and energy delivery (treatment) parameters may be displayed on the display 408. During energy delivery, the display 408 may be configured to display remaining treatment time, temperature, impedance, and power information (alphanumerically and/or graphically). For example, graphical representations of power vs. time and impedance vs. time may be displayed. In one implementation, the display may comprise a color, active matrix display. The generator 400 further includes a start/pause button 410 configured to be pressed by an operator to initiate and stop energy delivery. Similar to the indicator light 406 surrounding the instrument connection port 405, a second indicator light 412 may surround the start/pause button 410. The second indicator light 412 may also comprise a circular LED indicator light. The second indicator light 412 may be configured to continuously illuminate in a solid color (e.g., white, blue, green) when the generator 400 is powered on and ready to initiate energy delivery. The indicator light 412 may flash at a first pulsing rate (e.g., 1 Hz) to prompt a clinician to press the start/pause button 410 to initiate energy delivery. The indicator light 412 may flash at a second pulsing rate different than (e.g., faster than) the first pulsing rate (e.g., 2 Hz, 3 Hz, 4 Hz) when energy delivery has been paused or stopped. The generator 400 may also be configured to output audible alerts indicative of the different operating conditions (e.g., to coincide with the output of the indicator lights 406, 412.

The generator 400 may also include a power button 414 configured to power on and off the generator 400, a standby power indicator light 416 configured to illuminate (e.g., in solid green color) when an AC power switch (not shown) of the generator 400 is switched on, an RF active indicator light 417 configured to illuminate (e.g., in solid blue color) during RF energy delivery, and a system fault indicator light 418 configured to illuminate (e.g., in solid red color) during a system fault condition. The generator 400 may also include user input buttons 420 configured to facilitate navigation and selection of options (e.g., menu options, configuration options, acknowledgement requests) that appear on the display 408 (e.g., arrow buttons to toggle up and down between options and an “enter” button for user selection of a desired option).

### Access to Locations Outside the Vertebral Body

For access to locations outside bone (e.g., extraosseous locations, such as outside a vertebral body), visualization or imaging modalities and techniques may be used to facilitate targeting. For example, a foramen of a vertebral body (e.g., basivertebral foramen) may be located using MRI guidance provided by an external MR imaging system, CT guidance provided by an external tomography imaging system, fluoroscopic guidance using an external X-ray imaging system, and/or an endoscope inserted laparoscopically. Once the foramen is located, therapy (e.g., heat or energy delivery, chemoablative agent delivery, cryotherapy, brachytherapy, and/or mechanical severing) may be applied to the foramen sufficient to modulate (e.g., ablate, denervate, stimulate) any nerves entering through the foramen. For example, an endoscope may be used to locate the foramen under direct visualization and then the basivertebral nerve may be mechanically transected near the foramen. In some implementations, an intervertebral disc and vertebral body may be denervated by treating (e.g., ablating) a sinuvertebral nerve prior to the sinuvertebral nerve branching into the basivertebral nerve that enters the basivertebral foramen of the vertebral body. Because vertebral endplates are cartilaginous, radiation or high-intensity focused ultrasound energy may be applied to vertebral endplates from a location external to a subject’s body altogether to denervate nerves in the vertebral endplates.

### Target Identification and Patient Screening

In accordance with several implementations, target, or candidate, vertebrae for treatment can be identified prior to treatment. The target, or candidate, vertebrae may be identified based on identification of various types of, or factors associated with, endplate degeneration and/or defects (e.g., focal defects, erosive defects, rim defects, corner defects, all of which may be considered pre-Modic change characteristics). For example, one or more imaging modalities (e.g., MRI, CT, X-ray, fluoroscopic imaging) may be used to determine whether a vertebral body or vertebral endplate exhibits active Modic characteristics or “pre-Modic change” characteristics (e.g., characteristics likely to result in Modic changes, such as Type 1 Modic changes that include findings of inflammation and edema or type 2 Modic changes that include changes in bone marrow (e.g., fibrosis) and increased visceral fat content). For example, images obtained via MRI (e.g., IDEAL MRI) may be used to identify (e.g., via application of one or more filters) initial indications or precursors of edema or inflammation at a vertebral endplate prior to a formal characterization or diagnosis as a Type 1 Modic change. Examples of pre-Modic change characteristics could include mechanical characteristics (e.g., loss of soft nuclear material in an adjacent intervertebral disc of the vertebral body, reduced disc height, reduced hydrostatic pressure, microfractures, focal endplate defects, erosive endplate defects, rim endplate defects, corner endplate defects, osteitis, spondylodiscitis, Schmorl’s nodes) or bacterial characteristics (e.g., detection of bacteria that have entered an intervertebral disc adjacent to a vertebral body, a disc herniation or annulus tear which may have allowed bacteria to enter the intervertebral disc, inflammation or new capilarisation that may be caused by bacteria) or other pathogenetic mechanisms that provide initial indications or precursors of potential Modic changes or vertebral endplate degeneration or defects.

Accordingly, vertebral bodies may be identified as target candidates for treatment before Modic changes occur (or before painful symptoms manifest themselves to the patient) so that the patients can be proactively treated to prevent, or reduce the likelihood of, chronic low back pain before it occurs. In this manner, the patients will not have to suffer from debilitating lower back pain for a period of time prior to treatment. Modic changes may or may not be correlated with endplate defects and may or may not be used in candidate selection or screening. In accordance with several embodiments, Modic changes are not evaluated and only vertebral endplate degeneration and/or defects (e.g., pre-Modic change characteristics prior to onset or prior to the ability to identify Modic changes) are identified. Rostral and/or caudal endplates may be evaluated for pre-Modic changes (e.g., endplate defects that manifest before Modic changes that may affect subchondral and vertebral bone marrow adjacent to a vertebral body endplate).

In some implementations, a level of biomarker(s) (e.g., substance P, cytokines, high-sensitivity C-reactive protein, or other compounds associated with inflammatory processes and/or pain and/or that correlate with pathophysiological processes associated with vertebral endplate degeneration or defects (e.g., pre-Modic changes) or Modic changes such as disc resorption, Type III and Type IV collagen degradation and formation, or bone marrow fibrosis) may be obtained from a patient (e.g., through a blood draw (e.g., blood serum) or through a sample of cerebrospinal fluid) to determine whether the patient is a candidate for basivertebral nerve ablation treatment (e.g., whether they have one or more candidate vertebral bodies exhibiting factors or symptoms associated with endplate degeneration or defects (e.g., pre-Modic change characteristics)). Cytokine biomarker samples (e.g., pro-angiogenic serum cytokines such as vascular endothelial growth factor (VEGF)-C, VEGF-D, tyrosine-protein kinase receptor 2, VEGF receptor 1, intercellular adhesion molecule 1, vascular cell adhesion molecule 1) may be obtained from multiple different discs or vertebral bodies or foramina of the patient and compared with each other in order to determine the vertebral bodies to target for treatment. Other biomarkers may be assessed as well, such as neo-epitopes of type III and type IV pro-collagen (e.g., PRO-C3, PRO-C4) and type III and type IV collagen degradation neo-epitopes (e.g., C3M, C4M).

In some implementations, samples are obtained over a period of time and compared to determine changes in levels over time. For example, biomarkers may be measured weekly, bi-monthly, monthly, every 3 months, or every 6 months for a period of time and compared to analyze trends or changes over time. If significant changes are noted between the biomarker levels (e.g., changes indicative of endplate degeneration or defects (e.g., pre-Modic change characteristics) or Modic changes, as described above), treatment may be recommended and performed to prevent or treat back pain. Biomarker levels (e.g., substance P, cytokine protein levels, PRO-C3, PRO-C4, C3M, C4M levels) may be measured using various in vivo or in vitro kits, systems, and techniques (e.g., radio-immunoassay kits/methods, enzyme-linked immunosorbent assay kits, immunohistochemistry techniques, array-based systems, bioassay kits, in vivo injection of an anticytokine immunoglobulin, multiplexed fluorescent microsphere immune-assays, homogeneous time-resolved fluorescence assays, bead-based techniques, interferometers, flow cytometry, etc.). Cytokine proteins may be measured directly or indirectly, such as by measuring mRNA transcripts.

The identification of pre-Modic change characteristics may involve determining a quantitative or qualitative endplate score based on severity, extent, and/or quantity of the identified pre-Modic change characteristics (e.g., vertebral endplate defects) and vertebrae having a quantitative endplate score above a threshold may be deemed as potential candidates for treatment (e.g., basivertebral nerve ablation). The pre-Modic change characteristics may be combined with age, gender, body mass index, bone mineral density measurements, back pain history, and/or other known risk factors for vertebral endplate degeneration or defects (such as smoking, occupational or recreational physical demands or situations) in identifying candidate patients and/or candidate vertebral bodies for treatment (e.g., basivertebral nerve ablation).

### Conclusion

In some implementations, the system comprises various features that are present as single features (as opposed to multiple features). For example, in one embodiment, the system includes a single radiofrequency generator, a single introducer cannula with a single stylet, a single radiofrequency energy delivery device or probe, and a single bipolar pair of electrodes. A single thermocouple (or other means for measuring temperature) may also be included. Multiple features or components are provided in alternate embodiments.

In some implementations, the system comprises one or more of the following: means for tissue modulation (e.g., an ablation or other type of modulation catheter or delivery device), means for monitoring temperature (e.g., thermocouple, thermistor, infrared sensor), means for imaging (e.g., MRI, CT, fluoroscopy), means for accessing (e.g., introducer assembly, curved cannulas, drills, curettes), etc.

Although certain embodiments and examples have been described herein, aspects of the methods and devices shown and described in the present disclosure may be differently combined and/or modified to form still further embodiments. Additionally, the methods described herein may be practiced using any device suitable for performing the recited steps. Further, the disclosure (including the figures) herein of any particular feature, aspect, method, property, characteristic, quality, attribute, element, or the like in connection with various embodiments can be used in all other embodiments set forth herein. The section headings used herein are merely provided to enhance readability and are not intended to limit the scope of the embodiments disclosed in a particular section to the features or elements disclosed in that section.

While the embodiments are susceptible to various modifications, and alternative forms, specific examples thereof have been shown in the drawings and are herein described in detail. It should be understood, however, that the embodiments are not to be limited to the particular forms or methods disclosed, but to the contrary, the embodiments are to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “applying thermal energy” include “instructing the applying of thermal energy.”

The terms “top,” “bottom,” “first,” “second,” “upper,” “lower,” “height,” “width,” “length,” “end,” “side,” “horizontal,” “vertical,” and similar terms may be used herein; it should be understood that these terms have reference only to the structures shown in the figures and are utilized only to facilitate describing embodiments of the disclosure. The terms “proximal” and “distal” are opposite directional terms. For example, the distal end of a device or component is the end of the component that is furthest from the operator during ordinary use. A distal end or tip does not necessarily mean an extreme distal terminus. The proximal end refers to the opposite end, or the end nearest the operator during ordinary use. Various embodiments of the disclosure have been presented in a range format. It should be understood that the description in range format is merely for convenience and brevity and should not be construed as an inflexible limitation on the scope of the invention. The ranges disclosed herein encompass any and all overlap, sub-ranges, and combinations thereof, as well as individual numerical values within that range. For example, description of a range such as from 70 to 115 degrees should be considered to have specifically disclosed subranges such as from 70 to 80 degrees, from 70 to 100 degrees, from 70 to 110 degrees, from 80 to 100 degrees etc., as well as individual numbers within that range, for example, 70, 80, 90, 95, 100, 70.5, 90.5 and any whole and partial increments therebetween. Language such as “up to,” “at least,” “greater than,” “less than,” “between,” and the like includes the number recited. Numbers preceded by a term such as “about” or “approximately” include the recited numbers. For example, “about 2:1” includes “2:1.” For example, the terms “approximately”, “about”, and “substantially” as used herein represent an amount close to the stated amount that still performs a desired function or achieves a desired result.

1. A system for treating a vertebral body, comprising:
an introducer cannula having an introducer lumen disposed therethrough;
a curved cannula having an elongated tube with a curved cannula lumen disposed therethrough, the elongated tube having a proximal straight portion and a distal curved portion, the elongated tube configured to be received into the introducer lumen of the introducer cannula;
a stylet having an elongated shaft with a proximal straight portion and a distal curved portion, the elongated shaft configured to be received into the curved cannula lumen;
wherein the distal curved portion of the stylet comprises relief structures along an outer curvature configured to maintain a consistent cross-sectional profile during deflection; and
a treatment probe configured to be advanced through the curved cannula after removal of the stylet.

2. The system of claim 1, wherein the relief structures comprise a plurality of projections and recesses uniformly spaced along the outer curvature of the distal curved portion, wherein the plurality of projections and recesses are configured to create discrete flex points.

3. The system of claim 2, wherein the plurality of projections and recesses comprise a plurality of ridges radially-oriented and configured to engage with and support a curved cannula wall during deflection.

4. The system of claim 3, wherein each of the plurality of ridges has an edge profile to maintain smooth engagement with the curved cannula wall, wherein the edge profile is a filleted edge profile.

5. The system of claim 3, wherein each of the plurality of ridges are perpendicular to a longitudinal axis of the elongated shaft.

6. The system of claim 3, wherein the plurality of ridges have a depth configured to maintain structural integrity while enabling controlled bending.

7. The system of claim 1, wherein the distal curved portion includes a length proximal to the relief structures configured to prevent kinking.

8. The system of claim 1, wherein the stylet further comprises a distal channeling tip, wherein the distal channeling tip of the curved cannula has a sharper angle at the tip.

9. The system of claim 1, wherein the distal curved portion has a bend angle between 60 and 85degrees.

10. The system of claim 1, wherein the elongated shaft has a thickness between 0.05 and .06 inches at the distal curved portion.

11. The system of claim 1, wherein the relief structures extend along a length between 1.2 and 1.3 inches of the distal curved portion.

12. The system of claim 1, wherein the distal curved portion comprises an alignment feature having a semi-circular profile positioned at a proximal end of the curved portion.

13. The system of claim 1, wherein the treatment probe comprises a bipolar radiofrequency probe.

14. A medical device for accessing and navigating through bone, comprising:
a curved cannula comprising an elongated tube having a proximal straight portion, a distal curved portion, and a cannula lumen therethrough;
a stylet comprising an elongated shaft comprising a proximal handle portion, a straight shaft portion, and a distal curved portion;
wherein the distal curved portion comprises a plurality of ridges extending along an outer curvature;
wherein the plurality of ridges have filleted edges to facilitate insertion and removal through the curved cannula.

15. The device of claim 14, wherein the plurality of ridges are uniformly spaced.

16. The device of claim 14, wherein the distal curved portion includes a length proximal to the plurality of ridges configured to prevent kinking during insertion.

17. The device of claim 14, wherein each of the plurality of ridges are perpendicular to a longitudinal axis of the elongated shaft.

18. The device of claim 14, wherein the distal curved portion has a bend angle of at least 75 degrees.

19. The device of claim 14, wherein the plurality of ridges maintain a horseshoe cross-section when subjected to lateral forces during bone penetration.

20. A method for accessing a target location within a vertebral body, comprising:
inserting an introducer cannula through a pedicle adjacent to the vertebral body;
advancing a curved cannula assembly through the introducer cannula, wherein the curved cannula assembly comprises a curved cannula and a stylet, wherein the stylet comprises and elongated shaft having a distal curved portion with a plurality of circumferential ridges along an outer radius of the distal curved portion;
extending the curved cannula assembly beyond a distal end of the introducer cannula to create a curved path within the vertebral body, wherein the plurality of circumferential ridges maintain structural integrity during advancement through varying bone densities;
removing the stylet from the curved cannula; and
advancing a treatment device through the curved cannula to the target location.
